(19) United States
(12) Patent Application Publication
(10) Pub. No.: US 2026/0235573 A1
(43) Pub. Date: Aug. 13, 2026

(54) AQUATIC TOTAL ALKALINITY MEASUREMENT SYSTEM AND METHOD

(71) Applicant: PCFR, Cucuron (FR)

(72) Inventors: Lane Hoy, Vaugines (FR); Xavier Darok, Verquieres (FR); Sebastien Ettling Coeffier, Cabrieres d'Aigues (FR); Rene Brunier, Vaugines (FR)

(21) Appl. No.: 19/474,715

(22) PCT Filed: Apr. 12, 2024
(86) PCT No.: PCT/EP2024/060085

§ 371 Date: Oct. 12, 2025

(30) Foreign Application Priority Data
Apr. 13, 2023 (FR) FR2303675
Apr. 13, 2023 (FR) FR2303700
Jul. 25, 2023 (FR) FR2308017
Aug. 8, 2023 (FR) FR2308566

(51) Int. Cl. G01N 33/18; C02F 1/66; C02F 103/42; G01N 27/36; G01N 27/416

(52) U.S. Cl. CPC G01N 33/1886; C02F 1/66; G01N 27/36; G01N 27/4167; G01N 27/4168; G01N 33/182; C02F 2103/42; C02F 2209/04; C02F 2209/06; C02F 2209/07

(57) ABSTRACT

The aquatic total alkalinity measurement system (100) comprises:
a pH probe (105) configured to measure pH at the boundary layer of a body of water,
a probe controller (115), configured to sequentially activate and deactivate, or connect and disconnect, the pH probe,
a pH measurement variation detection device (120), configured to detect a variation of pH measurement in a sequence of pH probe measurements, and
an aquatic total alkalinity value determination device (125), configured to determine an aquatic total alkalinity value of the body of water as a function of the pH measurement variation detected, and
preferably a total alkalinity regulation unit, configured to increase or decrease the total alkalinity of the body of water as a function of the measured total alkalinity value and a target total alkalinity value.

### TECHNICAL FIELD OF THE INVENTION

The present invention relates to an aquatic total alkalinity measurement system and an aquatic total alkalinity measurement method. It applies, in particular, to the field of water treatment and to the field of in situ water total alkalinity measurement. The present invention applies to recreational aquatics (pools, spas, spray pads, water features, water fountains, water parks, wellness facilities, therapy facilities, lazy rivers, etc.) and to any similar industry or market segment where water is treated and/or monitored in a semi closed and/or closed circuit (such as waste water, water reuse, industrial water, drinking water, animal water, etc.) and to any similar industry or market segment where water is used as part of the process (such as evaporative cooling for energy generation and data centers, heating, ventilation and air conditioning, fire suppression stored water management, etc.).

The present invention also relates to an aquatic installation predictive maintenance system and an aquatic installation predictive maintenance method. It applies, in particular, to the field of physical and chemical water treatment and to the field of in situ physical and chemical water treatment. The present invention applies to commercial and residential recreational aquatics (pools, spas, spray pads, water features, water fountains, water parks, wellness facilities, therapy facilities, lazy rivers, etc.) and to any similar industry or market segment where water is treated and/or monitored in a semi closed and/or closed circuit (such as waste water, water reuse, industrial water, drinking water, animal water, etc.) and to any similar industry or market segment where water is used as part of the process (such as evaporative cooling for energy generation and data centers, heating, ventilation and air conditioning, and fire suppression stored water management, etc.).

The present invention also relates to an aquatic installation monitoring system and an aquatic installation monitoring method. It applies, in particular, to the field of water treatment and to the field of in situ water treatment. The present invention applies to recreational aquatics (pools, spas, spray pads, water features, water fountains, water parks, wellness facilities, therapy facilities, lazy rivers, etc.) and to any similar industry or market segment (evaporative cooling for energy generation and data centers, heating, ventilation and air conditioning, and fire suppression stored water management, etc.).

The present invention also relates to an aquatic installation four-dimensional monitoring system and an aquatic installation four-dimensional monitoring method. It applies, in particular, to the field of water treatment and to the field of in situ water treatment. The present invention applies to recreational aquatics (pools, spas, spray pads, water features, water fountains, water parks, wellness facilities, therapy facilities, lazy rivers, etc.) and to any similar industry or market segment (evaporative cooling for energy generation and data centers, heating, ventilation and air conditioning, and fire suppression stored water management, etc.).

### BACKGROUND OF THE INVENTION

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Total alkalinity is a key parameter of a body of water which significantly influences the capacity of chemical treatment to obtain a nominal state for that body of water.

Total alkalinity in water refers to the measure of the water's ability to neutralize acids. It is the total amount of alkaline substances (such as carbonates, bicarbonates, and hydroxides) present in the water and is expressed in milligrams per liter (mg/L) or parts per million (ppm). Maintaining proper total alkalinity levels in the water is crucial for ensuring the stability of pH (or “potential of hydrogen”) levels and preventing risk to users (such users could be bathers in a swimming pool with uncomfortable water creating skin irritation and/or health issues), damage to equipment, and/or damage to the containing structure itself.

pH has a significant influence on disinfection efficiency with many common disinfectants used or allowed in aquatics. When unstable increasing pH will dramatically and rapidly reduce disinfection efficiency, putting swimmers and facilities at risk.

Decreasing total alkalinity decreases pH stability, and increasing total alkalinity increases pH stability. A minimum total alkalinity level of 80 mg/l is generally considered to be a minimum for stable pH and predictable pH control and thus also disinfection regulation.

While total alkalinity can be measured with industrial equipment (typically by titration, or with the use of reagents) or even simple test strips, affordable simply real-time measurement does not exist because adding sensors and circulation control equipment adds complexity and cost generally deemed unsuitable in this application.

Currently, measurement of alkalinity is performed at a pH of 4.3, where bicarbonate (HCO₃⁻) turns into carbonic acid (H₂CO₃). Total alkalinity measures the amount of acid (H₃O⁺) needed to neutralize the bicarbonate (HCO₃⁻) in addition to carbonate (CO₃⁻) and hydroxide (OH⁻) in 100 ml of water. Currently, such systems require the use of reagents that are consumables. In current systems, total alkalinity measurement in water treatment is typically performed on site, using mono- or multi-parameter sensing devices (test strips or photo-colorimetric devices). Such systems require notable processing time, the use of expert and experienced labor, and dedicated tools and consumables.

In current digital aquatic installation control and monitoring systems, such as swimming pool control and monitoring systems, alerts associated with a need of maintenance of such installations are emitted as a function of the value of an operational parameter of an element associated to the aquatic installation and a corresponding alert threshold. Such an operational parameter may correspond to, for example, the need to change an empty consumable's installation, to clean the installation and/or the filter, or to maintain a circulation pump.

Such systems function on an element-by-element basis and are linear in their ways of functioning, considering they are limited to the comparison of an operational parameter value to a threshold to determine a need for intervention and/or maintenance.

However, operational parameters of aquatic facilities interact with each other, which renders linear control and monitoring systems inaccurate considering that an aquatic installation condition may deteriorate faster than anticipated by traditional and linear control and monitoring systems. To counteract such shortcomings, the alert threshold values are typically compensated, which leads to earlier than necessary alerts and, in the worst case, a lack of consideration by users for such alerts which may be deemed too conservative.

Furthermore, such systems typically stop at the emission of alerts, and the actual maintenance of the aquatic installation is processed outside of these systems. Therefore, the maintenance may or may not be performed, which in any case does not interact with these systems beyond the fact that monitored value may or may no longer trigger an alert if the maintenance of the cause of the alert was properly addressed.

All of these systems are thus unsatisfactory in regards of the timeliness and accuracy of aquatic installation maintenance, as well as in terms of capacity to monitor the state and maintenance operations linked with an aquatic installation or an ensemble of such aquatic installations, as well as in terms of capacity to manage the needs and use of consumable resources to maintain an ideal water state in compliance with user's and/or operator's set points (such as energy consumption, water consumption, and consumption for associated chemical treatment). For users in charge of dozens or hundreds of such aquatic installations, these problems compound to generate significant waste of maintenance operator time, as well as significantly increase the risk of maintenance requirements not being fulfilled on time, leading to health and safety risk to users and maintainers and permanent degradation of the aquatic installations, as well as significantly increasing the operating cost with an excess of needs and/or use of consumable resources to maintain an ideal water state in compliance with user's and/or operator's set points (such as energy consumption, water consumption, and consumption for associated chemical treatment).

In current systems, the efficiency of a water treatment circuit is measured within the hydraulic circuit (in the pipes, pumps and other chemical storing tanks) which supplies a water installation.

However, such systems are inefficient in that they do not account for the shape of the installation, the circulation of water within that installation, the external parameters (such as weather forecasts), number and sizes of bathers (in the case of swimming pool), pollution in the installation (such as leaves, algae spots or stain) and the surrounding of the installation. Therefore, in order to monitor the impact of a water treatment process, operators must test the chemical state of the water in the installation in a few locations to determine whether the treatment process is successful or not and report the external factors and/or parameters. Such constraints also apply to the diagnostics to define a water treatment process to be performed and adjusted based on the external factors and/or parameters influence. Furthermore, such monitoring is also instantaneous and thus only provides a snapshot of the physical and/or chemical state of an aquatic installation. Furthermore, such monitoring is static in terms of positioning and unadaptable to increase the quality of a measure of a particular physical/chemical value correlated to the real time need of the installation taking into account any external factors and/or parameters.

Therefore, current systems provide an inaccurate representation of the real-time and location dependent physical and/or chemical state and needs of water in an aquatic installation.

In current systems, the efficiency of a water treatment circuit is measured within the hydraulic circuit (in the pipes, pumps and other chemical storing tanks) which supplies an aquatic installation.

However, such systems are inefficient in that they do not account for the shape of the installation, the circulation of water within that installation, the external parameters (such as weather forecasts), number and sizes of bathers (in the case of swimming pool), pollution in the aquatic installation (such as leaves, algae spots or stain) and the surrounding of the installation. Therefore, in order to monitor the impact of a water treatment process, operators must periodically test the physical and chemical state of the water in the aquatic installation in multiple different locations to determine whether the treatment process is successful or not and report the external factors and/or parameters. Such constraints also apply to the diagnostics to define a water treatment process to be performed and adjusted based on the external factors and/or parameters influence. Furthermore, such monitoring is also instantaneous and thus only provides a snapshot of the physical and/or chemical state of an aquatic installation. Furthermore, such monitoring is static in terms of positioning and unadaptable to increase the quality of a measure of a particular physical/chemical value correlated to the real time need of the aquatic installation taking into account any external factors and/or parameters.

Therefore, current systems provide an inaccurate representation of the real-time and location dependent physical and/or chemical state and needs of water in an aquatic installation.

### SUMMARY OF THE INVENTION

The present invention aims at overcoming the above-mentioned drawbacks as well as other drawbacks that could be overcome although not mentioned in the description below.

The inventors have discovered that a discontinuous measurement of pH, in a body of water, using a probe and a floating reference (sometimes called “solution earth” or “liquid junction”) results in a variation of the pH measured, and that the quantification of this variation is tied to the total alkalinity value of the body of water. Hence, the present invention allows for the measurement of the total alkalinity value of a body of water without using an independent, complex, and costly total alkalinity measurement device or technique.

Such an invention does not require the use of reagents or consumables. Furthermore, such an invention does not require the water to be at a specific pH.

The present invention provides a practical and simple way of measuring total alkalinity in water treatment using a specific probe in contact with water. This in situ data measurement method can be combined with cloud-based data management and algorithms (including the use of artificial intelligence and/or machine learning) to create predictive metrics representative of the evolution of water parameters.

The in situ measurement probe can be placed in an analysis chamber, or in a pipe where water flows, or in a device inside the water. This allows a real time and in situ measurement without need for human intervention. The use of cloud-based data management allows users to have access to the total alkalinity values and behavior without being physically on site. All the data can be recorded to create a measurement log, and based on predictive algorithm, alerts may be sent to users with suitable recommended actions to adjust total alkalinity and water balance depending on the predicted risks level. Alternatively, regulation of total alkalinity can be achieved with algorithmic control of suitable feeder or dosing equipment.

Using such an invention, the following benefits can be achieved:
reliable electronic measurement of water parameters which are traditionally negatively affected by commonly occurring electrical currents and charge in water; these currents are always present due water/pipe friction and water turbulence cause by connectors, elbows, pump, filter,
in particular embodiments, for more accurate, stable and reliable pH measurement, the use of floating reference technology (sometimes called “solution earth” or “liquid junction”) could be optionally added and thus ensures that the probe and measurement electronics equipotential with the water being sampled, and thus eliminating the impact of any electrical charge and the result allow having smaller variations in measured pH to be used to determine total alkalinity,
measuring pH periodically (rather than constantly) allows for the detection of a correlation between pH change and total alkalinity:
   repeated periodic electronic measurement of pH in the same water causes change in measured pH,
   the amplitude of this measured pH change is proportional to the total alkalinity in the water sample, and
   the amplitude of pH change over multiple pH measurements can be used to deduce and derive a measure of total alkalinity quantitatively.
measuring pH periodically (rather than constantly) allows for the extension of the probe lifetime and for the reduced requirement for calibration action and thus lower maintenance and/or use of less skilled technicians.

The inventors have discovered that using a trained machine learning model to predict a risk of occurrence of a negative and/or damaging event and/or of an increase of consumable use and/or need (such as energy consumption, water consumption, and consumption for associated chemical treatment) in an aquatic installation, either relative to the water in the installation and/or to the installations itself and/or to the installed equipment, associated with a maintenance job scheduling capacity, allow for proactive and time-optimized and/or cost-optimized maintenance, and health and safety risk to users and maintainers.

Such a machine learning model may be trained on a variety of data, both internal to the water or the installation and external.

Such data may originate from a variety of sensors and data sources.

In particular, such sensors may be embedded into installed equipment, and floating and/or submersible mobile vehicles, that operate within aquatic installations.

In particular, such sensors may correspond to specific total alkalinity measurement devices.

In particular, such data sources may correspond to network and internet databases and data sources.

The inventors have discovered that using a combination of at least one physical/chemical sensor and/or at least one optical sensor and/or at least one installation external factors and/or parameters, allows for the accurate determination of the state and the needs of an aquatic installation.

Furthermore, the inventors have discovered that using an autonomous vehicle, configured to sense at least one parameter representative of the physical and/or chemical state of a body of water in proximity to the vehicle and associate such a value with a timestamp, both the local and global physical and/or chemical states of water in an aquatic installation and/or external factors and/or parameters management may be accurately monitored.

Such a monitoring system may further comprise an optical sensor which provides alternative data points to be used to reinforce the evaluation of the state of the water in an installation or the state of the installation itself. Such an optical sensor may also provide data points to be used in combination with physical and/or chemical sensors to accurately determine the state of the water in an installation or the state of the installation itself.

Such a monitoring system may be integrated in a feedback loop associating the autonomous vehicle, external factors and/or parameters management and a hydraulic system associated with the aquatic installation.

Such a monitoring system may be associated with advanced computation capabilities, such as by using machine learning, to provide an accurate representation of the physical and/or chemical state of the water and/or diagnostics of water treatment processes to be performed on said body of water.

Such a monitoring system may ease facility management in a variety of contexts, such as aquatic facilities for example.

The inventors have discovered that using an autonomous vehicle, configured to sense at least one parameter representative of the physical and/or chemical state of a body of water in proximity to the vehicle and associate such a value with a timestamp, both the local and global physical and/or chemical states of aquatic installation in an installation may be accurately monitored.

Such a monitoring system may be derived by adding various physical and/or chemical state computation means, based on the values sensed by the autonomous vehicle.

Such a monitoring system may be integrated in a feedback loop associating the autonomous vehicle and a hydraulic system associated with the aquatic installation.

Such a monitoring system may be associated with advanced computation capabilities, such as by using machine learning, to provide an accurate representation of the physical and/or chemical state of the water and/or diagnostics of water treatment processes to be performed on said body of water.

Such a monitoring system may ease facility management in a variety of contexts, such as swimming pools for example.

### BRIEF DESCRIPTION OF THE DRAWINGS

Other advantages, purposes and particular characteristics of the invention shall be apparent from the following non-exhaustive description of at least one particular system and method object of this invention, in relation to the drawings annexed hereto, in which:

FIG. 1 represents, schematically, a first view of a particular embodiment of a system object of the present invention,

FIG. 2 represents, schematically, a second view of a particular embodiment of a system object of the present invention,

FIG. 3 represents, schematically, the impact of a high total alkalinity on the measured pH of a particular embodiment of a system object of the present invention,

FIG. 4 represents, schematically, the impact of a low total alkalinity on the measured pH of a particular embodiment of a system object of the present invention,

FIG. 5 represents, schematically and in the form of a flowchart, a first particular succession of steps of a method object of the present invention,

FIG. 6 represents, schematically and in the form of a flowchart, a second particular succession of steps of a method object of the present invention,

FIG. 7 represents, schematically, a third view of a particular embodiment of a system object of the present invention,

FIG. 8 represents, schematically, a graph representing a succession of pH measurements at the boundary layer of a body of water for different total alkalinity values,

FIG. 9 represents, schematically, a particular embodiment of a system object of the present invention,

FIG. 10 represents, schematically and in the form of a flowchart, a particular succession of steps of a method object of the present invention,

FIG. 11 represents, schematically, a particular embodiment of a submersible vehicle used in the system object of the present invention,

FIG. 12 represents, schematically, a particular embodiment of a floating vehicle used in the system object of the present invention,

FIG. 13 represents, schematically and in the form of a flowchart, a particular succession of steps of a method object of the present invention,

FIG. 14 represents, schematically, a computing system capable of performing a method object of the present invention,

FIG. 15 represents, schematically, a particular embodiment of a system object of the present invention,

FIG. 16 represents, schematically and in the form of a flowchart, a particular succession of steps of a method object of the present invention,

FIG. 17 represents, schematically, a particular embodiment of a system object of the present invention, and

FIG. 18 represents, schematically and in the form of a flowchart, a particular succession of steps of a method object of the present invention.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

According to at least one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques or may include digital electronic devices such as at least one application-specific integrated circuit (ASIC) or field programmable gate array (FPGA) that is persistently programmed to perform the techniques or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

According to at least one embodiment, the present invention makes use of software, stored as instructions in a memory, ROM or storage that may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor. While each processor or core of the processor executes a single task at a time, computer system may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

It should be noted that the figures are not to scale.

It should be noted that, below, the terms “boundary layer” refer to a layer of more or less stationary fluid (such as water or air) immediately surrounding an immersed object in relative motion with the fluid.

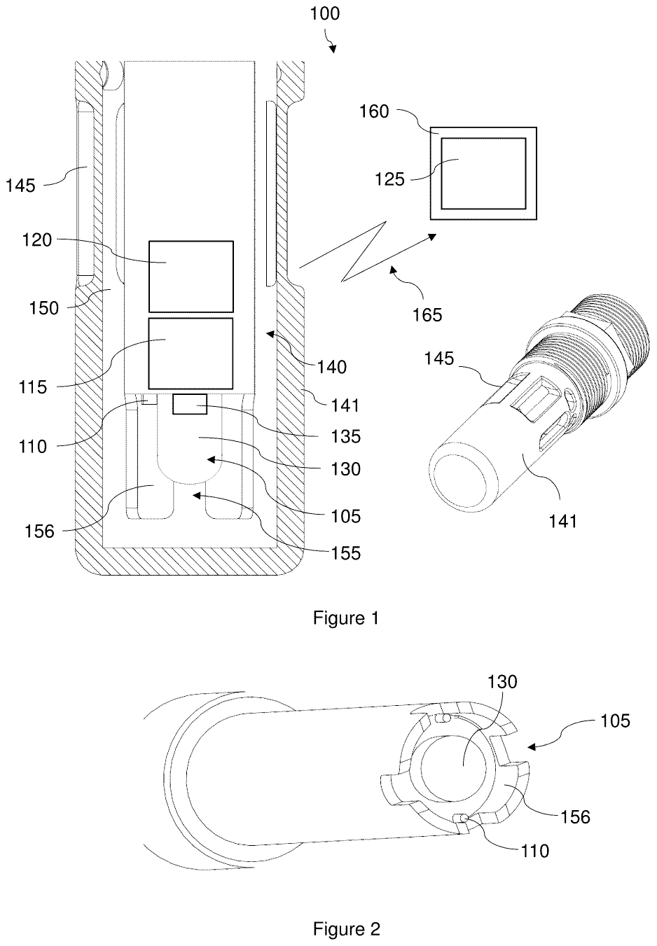

FIG. 1 represents, schematically, a particular embodiment of the system 100 object of the present invention. This aquatic total alkalinity measurement system 100, comprises:
a pH probe 105 configured to measure pH at the boundary layer of a body of water,
optionally, a floating reference device 110 in proximity of the pH probe,
a probe controller 115, configured to sequentially activate and deactivate the pH probe,
a pH measurement variation detection device 120, configured to detect a variation of pH measurement in a sequence of pH probe measurements, and
an aquatic total alkalinity value determination device 125, configured to determine an aquatic total alkalinity value of the body of water as a function of the pH measurement variation detected.

The pH probe 105 can be of any type known to a person skilled in the art that is suited for the particular implementation and intended use of the system 100. Such a pH probe 105 may differ in nature depending on the context of use of the system 100. For example, in a swimming pool, the pH probe 105 may comprise an oxidation-reduction potential sensor 135.

The objective of the pH probe 105 is to allow for the reproductible measurement of the pH in a body of water. Such a pH probe 105 is typically electronic and requires the supply of electrical energy to function. Such a pH probe 105 may further comprise a digital switch, allowing for the selective activation/deactivation of at least part of the core components of the pH probe 105.

The pH probe 105 may be mechanically located at the distal end of a sensor body, such as shown in FIGS. 1 to 4. The purpose of such a sensor body is the insertion in the body of water and, in preferred embodiments, within an analysis chamber 140.

The reference floating device 110, sometimes called “solution earth” or “liquid junction”, can correspond to any electrically conductive electrode or pin configured to normalize the signal sensed by the pH probe 105, avoiding electric noise in the proximity of the pH probe 105.

In the example shown in FIGS. 1 to 4, the reference floating device 110 comprises two electrodes, each located on a different side of a sensor 135 of the probe 105. Such electrodes may be diametrically opposed, with the sensor 135 acting as the center of a circle in which both electrodes are located on the periphery of said circle, for example. Such electrodes and the sensor 135 may be geometrically aligned.

In particular embodiments, such as the one shown in FIG. 1, the pH probe 105 comprises:
the floating reference device 110,
a microporous glass bulb membrane 130, and
an oxidation-reduction potential sensor 135.

pH measurement is based on the relationship between the concentration of H⁺ ions in tested water and the difference in electrochemical potential which is established in the lead-free glass bulb membrane of the probe. This lead-free bulb membrane is specifically designed to be selective to H⁺ ions concentration.

In general, the pH probe 105 is made of a simple electronic amplifier and a combined electrode, which consists of two electrodes: one whose potential is known and constant and the other whose potential varies with the pH.

Once the probe 105 is in contact with water, the H⁺ ions exchange on the glass bulb, creating an electrochemical potential across the bulb. The electronic amplifier detects the difference in electrical potential between the two electrodes generated in the measurement and converts the potential difference to pH units.

The pH value is determined by correlation because the potential difference between the two electrodes evolves proportionally to the pH according to the Nernst equation.

The probe controller 115 is, for example, an electronic circuit configured to electrically or electronically activate and deactivate, or connect and disconnect, the pH probe 105 or the sensor of said pH probe 105. Such an activation/deactivation or connection/disconnection may be performed by cutting and restoring power supply to the pH probe 105 or sensor or by emitting an activation/deactivation or connection/disconnection command to said pH probe 105 or sensor or relay.

The terms “activate and deactivate” relate to any hardware or software level activation/deactivation and/or to the connection/disconnection of the pH probe 105.

The probe controller 115 may itself be activated as a function of a command emitted by a computing device, located on site and mechanically connected to the pH probe 105 and/or the probe controller 115 or remotely located and connected to the probe controller 115 by way of a data connection.

The probe controller 115 may comprise, for example, a computer software executed upon a computing device, said computer software triggering the activation/deactivation or connection/disconnection of the pH probe 105. Such a computer software may correspond, for example, to a particular firmware or driver. Such a computer software may be updated remotely, and such an update may be automatically installed in the system 100.

The probe controller 115 may be configured to activate or connect the pH probe 105 periodically. The pH probe may be activated or physically connected, for example, every 60 seconds. Such an activation or physical connection may be conditional, for example to the activation of a water displacement pump. The rate of measurement may be variable depending on a mode configured. The duration of measurement may depend on the water's stability, so the pH measurement last until the measured pH is sufficiently stable.

Such an activation/deactivation can be performed by an electronic relay.

The pH measurement variation detection device 120 is, for example, an electronic device associated with the pH probe 105, configured to record a succession of pH values measured by the pH probe 105 and to compute, from said succession, a measurement variation value. Such a measurement variation value may be computed by the subtraction of a recent value from an older value.

The measured variation may be performed on immediately subsequent measured pH values or be sampled according to a particular sampling rule. Such variation may also be performed on an aggregate values of measured pH values.

For example, the pH measurement variation detection device 120 may be configured to subtract the average measured pH value during a specific, more recent timeframe from the average measured pH value during a specific, older timeframe. For example, the pH measurement variation detection device 120 may be configured to compute determine a mathematical function fitting a succession of data points linking measured pH to time of measurement since an initial measurement. Such an example is shown in FIG. 8. In other examples, the pH measurement variation detection device 120 may be configured to store, in a memory, a succession of data points linking measured pH to time of measurement since an initial measurement.

The system 100 may further comprise a timestamping means, configured to associate a time of measurement to a sensed pH value by the pH probe 105.

The action of repeatedly measuring the pH in the same water sample induces variations in the measurement of pH, the magnitude of these variations being dependent on the total alkalinity of the water. Such a pH measurement variation detection device 120 may also correspond to a computer software executed upon a computing device.

The pH measurement variation detection device 120 may operate remotely from the pH probe 105. In such a case, the system 100 may further comprise a communication means 165 to transmit data from the pH probe 105 to the pH measurement variation detection device 120. In such a case, the pH measurement variation detection device 120 may correspond to a computer program executed by a computing server, accessible on the cloud, via a data network such as the Internet for example.

The aquatic total alkalinity value determination device 125 is, for example, an electronic device associated with the pH measurement variation detection device 120, configured to associate a total alkalinity value to the measured variation.

For example, the total alkalinity value determination device 125 may be configured to compute the derivative of a mathematical function fitting a succession of data points linking measured pH to time of measurement since an initial measurement. Such an example is shown in FIG. 8.

The total alkalinity value determination device 125 may be configured to associate, with specific or ranges of said derivatives, a specific or a range of total alkalinity value.

For example, in FIG. 8:
a first series 805 of pH measurements (Y-axis), at specific times (X-axis), measured in minutes, since an initial measurement, for a total alkalinity value of 220 mg/l,
a second series 810 of pH measurements (Y-axis), at specific times (X-axis), measured in minutes, since an initial measurement, for a total alkalinity value of 125 mg/l, and
a third series 815 of pH measurements (Y-axis), at specific times (X-axis), measured in minutes, since an initial measurement, for a total alkalinity value of 19 mg/l.

Obtaining such series linking pH to alkalinity value relationship can be performed by empirically measuring, for different values of total alkalinity and a determined activation/connection frequency for the pH sensor, values of pH in the boundary layer of a body of water and storing these series in a memory. The number of such tests to be performed is limited in terms of scope, considering the limited number of values for alkalinity.

Such a total alkalinity value may be a mathematical function of the measured variation. Such a mathematical function may be performed by determining a regression function based upon the pH series captured, or derivative values of these series, as well as the operational parameters associated with the capture.

Such derivative values may be, for example, any type of averages or parameters of derivative functions.

For example, the following mathematical formula may be used (with initial parameters values: pH=7.4; water temperature=20° C.; ORP=700 mV; flow rate=0 m³/h):

$$A l k = - 0.0001 ( A V {G}_{1} - A V {G}_{2} ) + 0.1468$$

Where:
Alk designates the total alkalinity value,
AVG₁ designates the average pH values measured from 20 seconds to 80 seconds after the initial measurement, and
AVG₂ designates the average pH values measured from 300 seconds to 360 seconds after the initial measurement.
Such a function may be approximated to Alk=(AVG₁−AVG₂).

From such a function, and initial parameters, the following correspondence table may be obtained:

| Total alkalinity value | AVG1-AVG2 |
|---|---|
| 10 | 0.1368 |
| 20 | 0.1268 |
| 30 | 0.1168 |
| 40 | 0.1068 |
| 50 | 0.0968 |
| 60 | 0.0868 |
| 70 | 0.0768 |
| 80 | 0.0668 |
| 90 | 0.0568 |
| 100 | 0.0468 |
| 110 | 0.0368 |
| 120 | 0.0268 |
| 130 | 0.0168 |
| 140 | 0.0068 |
| 150 | −0.0032 |
| 160 | −0.0132 |
| 170 | −0.0232 |
| 180 | −0.0332 |
| 190 | −0.0432 |
| 200 | −0.0532 |

Such a total alkalinity value may be determined as a function of the measured variation and a preset threshold value, representative of a particular total alkalinity value.

Such an aquatic total alkalinity value determination device 125 may also correspond to computer software executed upon a computing device.

The aquatic total alkalinity value determination device 125 may operate remotely from the pH probe 105 and/or the pH measurement variation detection device 120. In such a case, the system 100 may further comprise a communication means 165 to transmit data from the pH measurement variation detection device 120 to the aquatic total alkalinity value determination device 125. In such a case, the aquatic total alkalinity value determination device 125 may correspond to a computer program executed by a computing server, accessible on the cloud, via a data network such as the Internet for example.

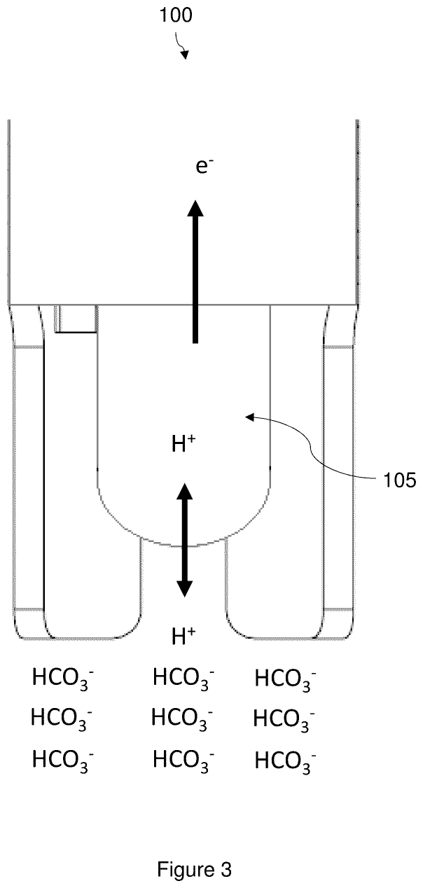

FIG. 3 shows the impact of a high total alkalinity body of water upon the system 100 object of the present invention.

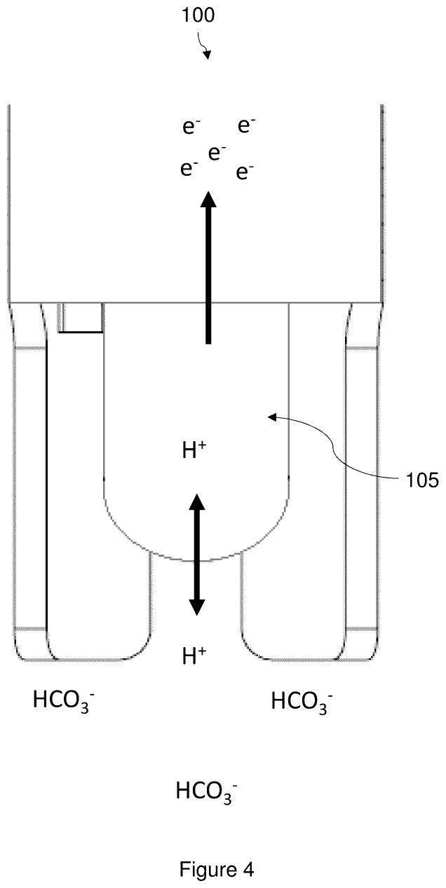

FIG. 4 shows the impact of a low total alkalinity body of water upon the system 100 object of the present invention.

In particular embodiments, the pH probe controller 115 is configured to sequentially activate and deactivate, or connect and disconnect, the pH probe 105 in a body of water with no flow. Such a state may be reached by stopping a pumping system introducing water in the body of water. In particular variants, the pH probe 105 may be activated after an absence of flow is detected (by a flow sensor, for example). In particular variants, a chamber in which the pH probe 105 is located may comprise valves that may be closed prior to the operation of the pH probe 105 activation/deactivation or connection/disconnection sequence.

The terms “body of water with no flow” designate a body of water with limited water flowing. In such a body of water, the water may circulate, but limited new water may enter.

In particular embodiments, the pH probe 105 is configured to be positioned in a small-volume body of water. Such a small volume may correspond to, for example, 1 to 2 milliliters.

The terms “small-volume body of water” designate a body of water in which the chemical reaction taking place during an interval of deactivation/activation, or connection/disconnection, of the pH probe 105 provides significant impact on the pH measure so as to show a variation between two successive measurements of the pH by the pH probe 105.

In particular embodiments, the system 100 object of the present invention comprises an analysis chamber 140, comprising an opening 145, a main volume 150 connected to the opening 145 and a recess 155 in the main volume 150, the pH probe 105 being in contact with the water in the recess 155.

The analysis chamber 140 may comprise a sensor housing 141 delimiting an internal volume in which the pH probe 105 or a sensor body associated with said pH probe 105 may be inserted.

The analysis chamber 140 is preferably configured to limit the flow of water and the volume of water in proximity to the pH probe 105. Such a configuration may be performed by selecting dimensions that limit the quantity of water entering the analysis chamber 140.

The analysis chamber 140 comprises an opening 145, of arbitrary dimensions, which allows for the passage of water from the body of water to the proximity of the pH probe 105.

The analysis chamber 140 comprises a main volume 150, defined for example by the interior dimensions of the sensor housing 141.

The analysis chamber 140 comprises a recess 155, defined by a subset of the interior dimensions of the sensor housing 141. In particular embodiments, the recess 155 is formed by crenellated sensor body extensions 156 associated to the pH probe 105, said crenellated sensor body extensions 156 limiting the movement of water in the proximity of the pH probe 105.

There are many possible configurations of the analysis chamber 140. Such configurations preferably limit the quantity of water in proximity to the pH probe 105 and/or limit the movement of water in proximity to the pH probe 105.

In particular embodiments, the system 100 object of the present invention comprises a remote computing device 160 comprising the aquatic total alkalinity value determination device 125 and a communication means 165 between the pH measurement variation detection device 120 and the aquatic total alkalinity value determination device 125.

Such a remote computing device 160 may correspond to, for example, a computing server hosted remotely and accessible through a data network, such as the Internet for example.

The communication means 165 is, for example, a communication interface coupled to bus. Communication interface provides a two-way data communication coupling to network link(s) that are directly or indirectly connected to at least one communication network, such as a network or a public or private cloud on the Internet. For example, communication interface may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network broadly represents a local area network (LAN), wide-area network (WAN), campus network, internetwork, or any combination thereof. Communication interface may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, the communication interface sends and receives electrical, electromagnetic, or optical signals over signal paths that carry digital data streams representing various types of information.

Network link typically provides electrical, electromagnetic, or optical data communication directly or through at least one network to other data devices, using, for example, satellite, cellular, Wi-Fi, or BLUETOOTH technology. For example, network link 265 may provide a connection through a network to a host computer.

Furthermore, network link may provide a connection through network or to other computing devices via internetworking devices and/or computers that are operated by an Internet Service Provider (ISP). ISP provides data communication services through a world-wide packet data communication network represented as the internet. A server computer may be coupled to the internet. Server broadly represents any computer, data center, virtual machine, or virtual computing instance with or without a hypervisor, or computer executing a containerized program system such as DOCKER or KUBERNETES. Server may represent an electronic digital service that is implemented using more than one computer or instance and that is accessed and used by transmitting web services requests, uniform resource locator (URL) strings with parameters in HTTP payloads, API calls, app services calls, or other service calls. Computer system and server may form elements of a distributed computing system that includes other computers, a processing cluster, server farm or other organization of computers that cooperate to perform tasks or execute applications or services. Server may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server may comprise a web application server that hosts a presentation layer, application layer and data storage layer such as a relational database system using structured query language (SQL) or no SQL, an object store, a graph database, a flat file system or other data storage.

The communication means 165 can send messages and receive data and instructions, including program code, through the network(s), network link and communication interface. In the Internet example, a server might transmit a requested code for an application program through Internet, ISP, local network and communication interface. The received code may be executed by processor as it is received, and/or stored in storage, or other non-volatile storage for later execution.

In particular embodiments, the aquatic total alkalinity value determination device 125 operates an algorithm and/or a trained machine learning model to associate an aquatic total alkalinity value with a variation in measured pH.

In particular embodiments, the pH probe 105 is configured to measure the pH of the body of water in a swimming pool.

In particular embodiments, the pH probe 105 is configured to measure the pH of the body of water in a pipe.

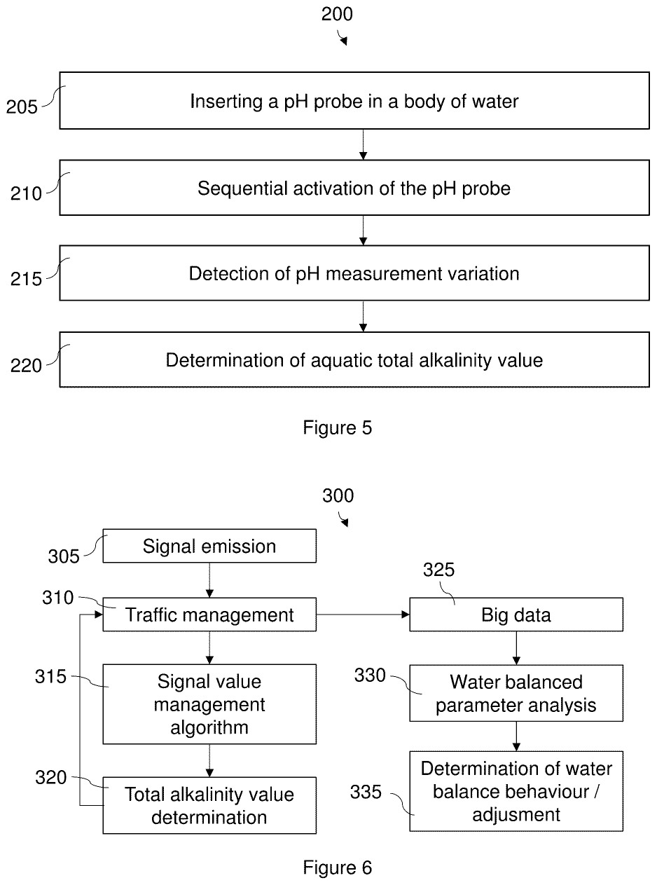

FIG. 5 shows, schematically, a particular embodiment of the method 200 object of the present invention. This aquatic total alkalinity measurement method 200 comprises:
a step 205 of inserting, in a body of water, a pH probe configured to measure pH at the boundary layer of a body of water,
a step 210 of sequential activation and deactivation, or connection and disconnection, of the pH probe,
a step 215 of detection of pH measurement variation, to detect a variation of pH measurement in a sequence of pH probe measurements, and
a step 220 of determination of an aquatic total alkalinity value, to determine an aquatic total alkalinity value of the body of water as a function of the pH measurement variation detected.

Embodiments of such a method 200 are disclosed in regard to FIGS. 1 to 3.

FIG. 6 shows, schematically, a particular embodiment of the method 300 object of the present invention. This method 300 comprises:
a step 305 of signal emission, by the pH probe 105 or the pH measurement variation detection device 120, of a measured pH or pH measurement variation,
a step 310 of traffic management, configured to address the measured pH or pH measurement variation to a specific software module and the associated hardware, and
in a first stream:
   a step 315 of execution of a signal value management algorithm, configured to extract values from the measured pH or pH measurement variation, and
   a step 320 of total alkalinity value determination, as a function of the signal value management algorithm executed,
in a second stream:
   a step 325 of big data analysis of the total alkalinity value,
   a step 330 of water balanced parameter analysis, and
   a step 335 of determination of water balance behavior/adjustments to be performed upon the body of water.

As it can be understood:

The use of an integrated floating earth, a microporous glass bulb membrane and an ORP sensor, placed in an analysis chamber, allows to provide accurate, reliable and continuous data measurement.

The core of such an analysis chamber preferably fits the sensor to limit the amount of water in the chamber part dedicated to measurement. When the water is flowing, water parameters are homogeneous in this chamber part. Once the flow stops, a chemicals parameters rate gradient occurs in this chamber part that does not happen, or in a limited fashion, in the global water in the analysis chamber. This is due, in that particular example, to the ionic exchange through the microporous glass bulb membrane of the probe between the core of the probe and a reactional volume close to the probe. Other configurations for the probe would yield similar results.

The inventors have discovered the existence of pH value micro variations (considered as hysteresis) when the water is not flowing in the analysis chamber and when the pH measurement stops at the same time. Those variations are directly linked to total alkalinity value in the analysis zone.

When the total alkalinity is high, shown in FIG. 3, a high concentration of HCO₃⁻ is located in the analysis zone. These ions absorb a major part of the pH micro variation linked to the chemical reaction between H⁺ in the water and H⁺ in the probe through the microporous glass bulb membrane. These micro variations are treated by the probe and generate a low electrical signal.

When the total alkalinity is low, shown in FIG. 4, a low concentration of HCO₃⁻ is located in the analysis zone. These ions absorb a minor part of the pH micro variation linked to the chemical reaction between H⁺ in the water and H⁺ in the probe through the micro porous glass bulb membrane. These micro variations are treated by the probe and generate a high electrical signal.

These electrical signals are preferably measured thanks to the probe being associated with the floating reference device, which allows the exclusion of any background hysteresis and the signal processing to focus on exact chemicals measurement.

As shown in FIGS. 3 and 4, the intensity of the measured signal is directly linked to the total alkalinity value. The higher the total alkalinity is, the lower will be the signal, and vice-versa.

These signals can be sent and treated in a cloud-based computing solution, such as shown in FIG. 6. Once the electrical signal is emitted by the probe, it is sent by a traffic manager to a dedicated cloud application and data storage. The traffic manager can transmit the data to a specific algorithm, where the signal value is transformed into a total alkalinity value. This value can be routed to a big data management device, using the traffic manager.

Further algorithms can use the total alkalinity value to compare it to the other water balanced parameters, in order to determine the water balance behavior and needed adjustment. For example, measured total alkalinity values, can be combined with water temperature values, total hardness values, total dissolved salt values, measured pH and desired pH values, disinfection chemical ppm values, oxidation-reduction potential values, and any other values applicable to the specific application. These combinations, used with the Langelier saturation index, can give the balance behavior of the water for measured and desired pH values, and also other chemical values that depend on balanced pH conditions. Thus, it can immediately be known if the water is balanced, aggressive or scaling, and also enables estimation of the water's future behavior and condition, depending on those values, modifications and trends.

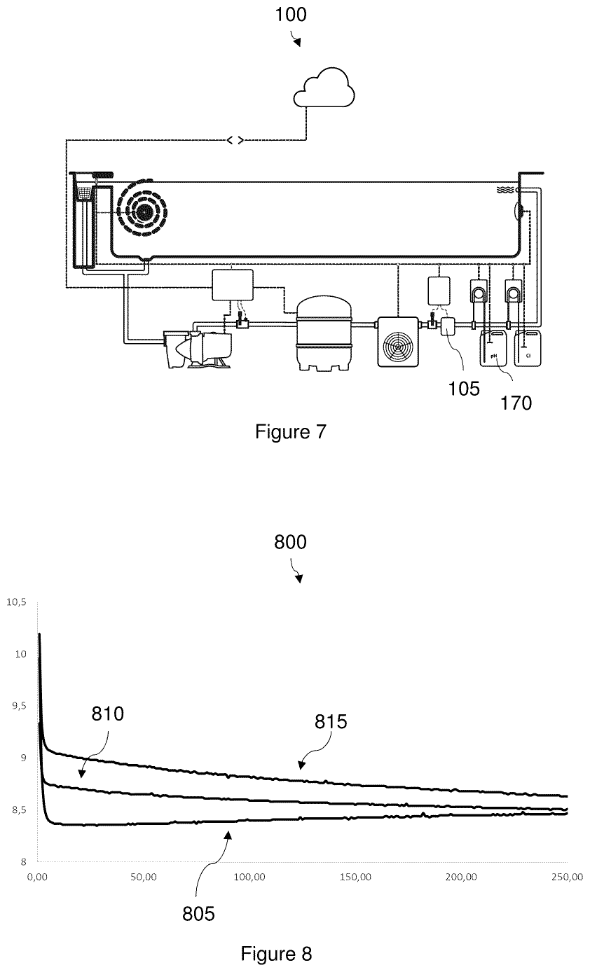

FIG. 7 represents a particular embodiment of the system 100 object of the present invention, which comprises a total alkalinity regulation unit 170, configured to increase or decrease the total alkalinity of the body of water as a function of the measured total alkalinity value and a target total alkalinity value.

The total alkalinity regulation unit 170 may correspond to any device suited for the regulation of alkalinity known to one skilled in the art. Such a device may be, for example, a mixer configured to mix acid from a reservoir and a stream of water processed in a swimming pool water treatment circuit.

The total alkalinity regulation unit 170 operates to reach a target total alkalinity in the body of water. This target total alkalinity can be set by a user or be remotely set by a computing system. Such a value may be higher or equal to 120 mg/l (or ppm) and lower than 250 mg/l (or ppm). If the measured total alkalinity is above the target total alkalinity, the regulation unit 170 may be operated to reduce the total alkalinity in the body of water whereas if the measured total alkalinity is below the target total alkalinity, the regulation unit 170 may be operated to increase the total alkalinity in the body of water.

### OBJECT OF THE INVENTION

The present invention is intended to remedy all or part of these disadvantages.

To this effect, according to a first aspect, the present invention aims at an aquatic total alkalinity measurement system, comprising:
a pH probe configured to measure pH at the boundary layer of a body of water,
a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

Such provisions allow for the accurate and near real-time measurement of the total alkalinity value of a body of water at an inexpensive cost and with ordinary equipment. However, the benefits of this invention result from the counter-intuitive discovery, by the inventors, that switching the pH probe on and off when the water is not flowing provides an accurate total alkalinity measurement whereas continuous pH measurement does not. This is because the successive activation/deactivation, or connection/disconnection of the pH probe results in a chemical reaction taking place in the vicinity of the pH probe. This chemical reaction results in a variation in pH measurement, said variation being dependent on the total alkalinity value of the water in the vicinity of the pH probe off, when the water is not flowing. Therefore, the present invention allows for the determination of the total alkalinity value of a body of water without using a total alkalinity measurement sensor. Such an indirect measurement significantly improves the capacity to measure total alkalinity in swimming pools and in any other aquatic installation and management systems.

In particular embodiments, the system object of the present invention comprises a floating reference device in proximity to the pH probe.

Such embodiments reduce the hysteresis of the pH probe.

In particular embodiments, the pH probe comprises:
the floating reference device,
a microporous glass bulb membrane, and
an oxidation-reduction potential sensor.

Such embodiments allow for an ionic exchange to take place in the vicinity of the microporous glass bulb membrane, said ionic exchange resulting in the measurement variations of the pH by the pH probe.

In particular embodiments, the pH probe controller is configured to sequentially activate and deactivate, or connect and disconnect, the pH probe in a non-flowing water.

The less the water in the body of water around the pH probe is renewed in between a deactivation and an activation, or the disconnection and connection, of the pH probe, the more representative of the total alkalinity of the water the variation between successive pH measurements is.

In particular embodiments, the pH probe is configured to be positioned in a low-volume body of water.

Such embodiments allow for the frequent activation/deactivation, or connection/disconnection, of the pH probe and thus more frequent measurement of total alkalinity of the body of water.

In particular embodiments, the system object of the present invention comprises an analysis chamber, comprising an opening, a main volume connected to the opening and a recess in the main volume, the pH probe being in contact with the water in the recess.

In particular embodiments, the system object of the present invention comprises a remote computing device comprising the aquatic total alkalinity value determination device and a communication means between the pH measurement variation detection device and the aquatic total alkalinity value determination device.

Such embodiments allow for the use of advanced calculation techniques in a centralized location, thus improving the quality of output of a fleet of pH probes.

In particular embodiments, the aquatic total alkalinity value determination device operates an algorithm and/or a trained machine learning model to associate an aquatic total alkalinity value with a variation in measured pH.

Such embodiments allow for the use of advanced calculation techniques in a centralized location, thus improving the quality of output of a fleet of pH probes.

In particular embodiments, the pH probe is configured to measure the pH of the body of water in a swimming pool and/or in an aquatic installation.

In particular embodiments, the pH probe is configured to measure the pH of the body of water in a pipe.

In particular embodiments, the system object of the present invention comprises a total alkalinity regulation unit, configured to increase or decrease the total alkalinity of the body of water as a function of the measured total alkalinity value and a target total alkalinity value.

According to a second aspect, the present invention aims at an aquatic total alkalinity measurement method, which comprises:
a step of inserting, in a body of water, a pH probe configured to measure pH at the boundary layer of a body of water,
a step of sequential activation and deactivation, or connection and disconnect, of the pH probe,
a step of detection of pH measurement variation, to detect a variation of pH measurement in a sequence of pH probe measurements, and
a step of determination of an aquatic total alkalinity value, to determine an aquatic total alkalinity value of the body of water as a function of the pH measurement variation detected.

The benefits of the method object of the present invention are similar to the benefits of the system object of the present invention.

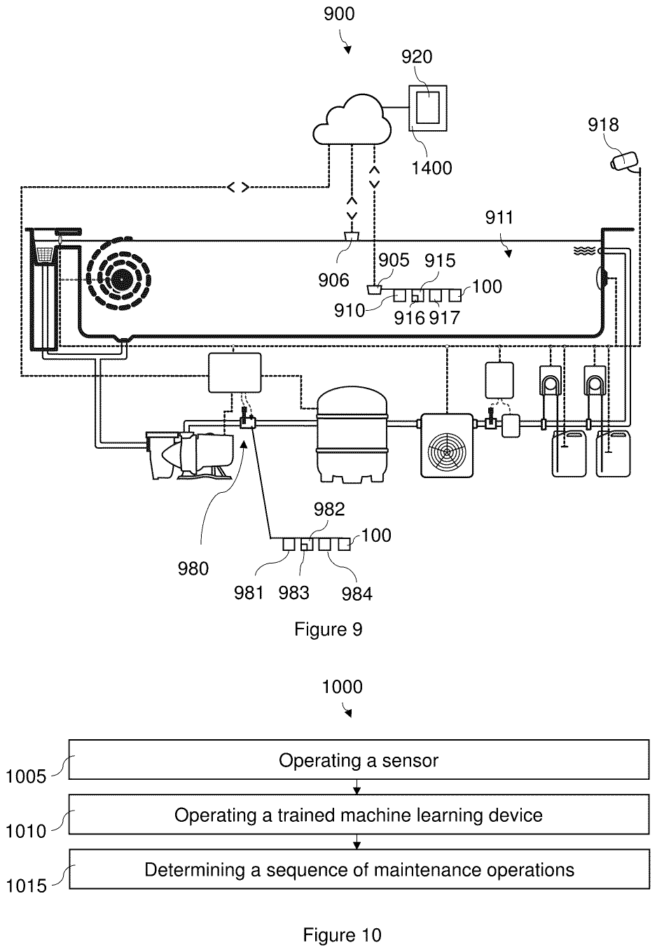

FIG. 9 represents, schematically, a particular embodiment of the system 900 object of the present invention. This aquatic installation predictive maintenance system 900 may comprise:
at least one physical/chemical sensor, 910, 915, 916, 917, 981, 982, 983, and 984, interacting with water in at least one aquatic installation and configured to provide series of at least one sensed value representative of a physical/chemical parameter, wherein, for example:
   at least one physical/chemical sensor, 981, 982, 983 and/or 984, is situated in an analysis chamber 980, or in a pipe of a circulation system where water flows, interacting with water in at least one aquatic installation and configured to provide series of at least one sensed value representative of a physical/chemical parameter and
   at least one physical/chemical sensor, 910, 915, 916, and/or 917, is situated in a submersible vehicle (ROV) 905 and/or a floating vehicle 906, interacting with water in at least one aquatic installation and configured to provide series of at least one sensed value representative of a physical/chemical parameter, and
at least one processor 920 configured to execute instructions representative of the steps of:
   operating a trained machine learning model, said model being trained to associate, for at least one series of sensed value representative of a physical/chemical parameter, at least one aquatic installation operational degradation event associated with a date of event occurrence, and
   determining a sequence of maintenance operations to be performed on at least one aquatic installation as a function of at least one predicted aquatic installation operational degradation event and associated date of event occurrence.

The physical and/or chemical sensor, 910, 915, 916, 917, 981, 982, 983 and/or 984, is intended in the broadest sense, meaning that any physical and/or chemical parameter sensing device is encompassed, provided the output data of such a device is used to evaluate the physical and/or chemical state of the water in a installation 911 and/or the physical and/or chemical state of the installation 911, and/or status of equipment in the aquatic installation.

Such a physical and/or chemical sensor, 910, 915, 916, 917, 981, 982, 983 and/or 984, is configured to sense a value of a physical and/or chemical parameter at a specific time, allowing for a succession of values sensed to be associated into a series. Such an association may be performed by the physical and/or chemical sensor, 910, 915, 916, 917, 981, 982, 983 and/or 984, or by a computing device receiving a succession of data representative of the values sensed by said physical and/or chemical sensor, 910, 915, 916, 917, 981, 982, 983 and/or 984.

Such a physical and/or chemical sensor, 910, 915, 916, 917, 981, 982, 983 and/or 984, may correspond to but is not limited to:
a pH sensor, and/or
a total alkalinity sensor, and/or
a conductivity sensor, and/or
an oxidation-reduction potential sensor, and/or
a turbidity sensor, and/or
an optical sensor, and/or
a camera and/or video camera, and/or
an acoustic and/or sonar sensor, and/or
a temperature sensor, and/or
a flow sensor, and/or
a water movement sensor, and/or
a pressure sensor.

In particular embodiments, such as the one shown in FIG. 9, at least one physical/chemical sensor, 910, 915, 916, 917, 918, 981, 982, 983 and/or 984, is associated with geographical coordinates, the step 1015 of determining a sequence being configured to further determine a sequence as a function of geographical coordinates of aquatic facilities associated with at least one predicted aquatic installation operational degradation event.

This sensor to geographical coordinates may be pre-set by a user or automatically determined by using a geolocation device, such as a GPS sensor, and/or accelerometer, for example.

In particular embodiments, such as the one shown in FIG. 9, the system 900 object of the present invention comprises at least one physical/chemical sensor is an aquatic total alkalinity measurement device such as shown in FIG. 1.

The vehicle 905 may correspond to a remotely or autonomously dirigible submarine vehicle, for example, such as shown in FIG. 11.

The vehicle 906 may also correspond to a floating device, such as shown in FIG. 12.

In particular embodiments, such as the one shown in FIG. 9, the system 900 comprises both a submersible vehicle (ROV) 905 and a floating vehicle 906.

In particular variants, at least one submersible and/or floating vehicle, 905 and/or 906, comprises a solar panel 305 configured to power an autonomous electricity source (not represented) and/or to charge the onboarded batteries (not represented).

In particular variants, at least one submersible and/or floating vehicle, 905 and/or 906, comprises an induction current collector configured to power an autonomous electricity source (not represented).

In particular variants, at least one submersible and/or floating vehicle, 905 and/or 906, comprises a power inlet configured to be connected to a charging wire to power an autonomous electricity source (not represented).

In particular variants, at least one submersible and/or floating vehicle, 905 and/or 906, comprises a propulsion 310 system, such as an engine associated with a boat propeller. Such a propulsion 310 system allows for the vehicle, 905 and/or 906, to move around in the water of the installation 911. This propulsion system 310 may comprise rear propellers 320, configured to generate forward, backward or yaw movements, and a front propeller 315, configured to generate upward or downward movements.

Such a submersible and/or floating vehicle, 905 and/or 906, may further comprise a relative positioning coordinates acquisition means, configured to locate, in a three-dimensional space representative of the aquatic installation 911, the submersible vehicle, 905 and/or 906, and to provide the corresponding coordinates of the submersible and/or floating vehicle, 905 and/or 906.

Such a relative positioning coordinates acquisition means is, for example, an acoustic and/or sonar configured to provide distance values from edges of the installation 911. The distance values allow for the determination of the shape of the installation 911. Once the shape of the installation 911 is known, such distance values allow for the determination of the positioning of the submersible and/or floating vehicle, 905 and/or 906, within said installation 911.

In another variant, the relative positioning coordinates acquisition means is, for example, a mechanical sensor used in coordination with a propulsion system 310 to map the shape of the installation 911 by detecting collisions of the submersible and/or floating vehicle, 905 and/or 906, with the edges of this installation 911.

Once the shape of the installation 911 is known, information originating from original parameters of the propulsion system 310 may be used to locate the submersible and/or floating vehicle, 905 and/or 906. For example, a duration of use of the propulsion system 310, associated with a power of propulsion, may be used in a calculation to determine a distance of the submersible and/or floating vehicle, 905 and/or 906, from the last known location.

The data resulting from the physical and/or chemical sensing means 910 and the relative positioning coordinates acquisition means may be aggregated to form a timestamped water physical and/or chemical value sensed. This data may further be associated with environmental context values, such as water pressure or time of capture for example.

In particular variants, the submersible and/or floating vehicle, 905 and/or 906, comprises an aquatic installation local physical and/or chemical state information aggregation means.

The aquatic installation local physical and/or chemical state information aggregation means is, for example, a computer software executed upon a computing device. This computing device is configured to associate, in a memory, the data resulting from the relative positioning coordinates acquisition means, the physical and/or chemical sensing means 910 and a timestamping means.

Such an association may be performed by concatenating said data in a single data stream or data frame or by creating a link between said data if such data is stored in separate database tables for example.

The timestamping means may correspond, for example, to any electronic clock used by a computing device. Such a timestamping means may be integrated into the submersible and/or floating vehicle, 905 and/or 906, or be remotely located from said submersible and/or floating vehicle, 905 and/or 906. By remotely located, it is intended that the timestamping means is linked with the submersible and/or floating vehicle, 905 and/or 906, by a communication means, such as a peer-to-peer link or a communications network link, such as the Internet for example.

The submersible and/or floating vehicle, 905 and/or 906, may further comprise an optical sensor 915, configured to provide a graphical representation of the water and/or aquatic installation, said representation being used during the step of operating the trained machine learning model.

Such an optical sensor 915 corresponds, for example, to a camera or to a video camera configured to capture images of the installation 911 and/or the water in the installation 911. In particular embodiments, this camera and/or video camera can be used to capture pictures and/or videos of the floor and walls of an aquatic reservoir to detect spots and define their origin and/or nature based on the color, location and form of said spots. In particular embodiments, this camera and/or video camera can be used to determine the water transparency and/or turbidity by analyzing the acquisition resolution and or restitution of a specific target point and/or target device location in a specific place in the pool. In particular embodiments, this target point and/or target device could be the inductive charging plate, and/or the installation's walls and/or floor, and/or a dedicated device placed in a defined location.

In particular embodiments, such as the one represented in FIG. 9, the optical sensor 915 comprises an infrared sensor 916. In particular embodiments, the infrared sensor 916 can allow the acquisition of thermic images, allowing the determination of an evolution of a water temperature evolution and/or differences within the installation. In particular embodiments, said temperatures evolution and/or differences can be registered and associated to the location to create a temperature mapping according to three axes according to times of measurement. In particular embodiments, this map can be used to adjust the water temperature homogeneity in the installation by, for example, adjusting the flow rate of the filtration pump and/or the filtration operating time and/or the operating time of the heat pump and/or the temperature set point of the heat pump and/or filter cleaning process, by increasing or decreasing their values to reach the targeted temperature homogeneity in the installation.

In particular embodiments, such as the one represented in FIG. 9, the submersible and/or floating vehicle, 905 and/or 906, comprises an acoustic and/or sonar sensor 917.

Based on the sound modifications perceived by the acoustic and/or sonar sensor, notifications and/or alerts can be emitted and/or equipment operating set point can be adjusted automatically.

For example, a pump which starts operating provides a specific sound (vibrating signature), these vibrations follow the water flow into the installation 911. Therefore, the use of an acoustic and/or sonar sensor allows for the measurement and analysis of these vibrations. Any change in this signature thus results from an interaction and/or a problem which has occurred between the pump and the installation 911. Such an interaction may correspond to a non-activation of the pump, a pump surge, a pump cavitation, an object/pollution in the pipe, a leak or a product injection. By measuring a difference between the normal state sound and the current measured sound, the diagnostic can be made, and/or notifications and/or alerts can be emitted and/or equipment operating set point can be adjusted automatically. This difference can also be linked to other types of data collected.

Any sound that may occur in the installation 911 can be linked to a type, or class, of event. The determination of this class can be obtained by using a trained machine learning classifier model. Such a trained machine learning classifier model can be obtained by feeding, in a machine learning classifier device, a sample comprising sounds and the related events. Such a trained machine learning classifier model can be obtained by feeding, in a machine learning classifier device, a sample comprising the sound in installations in the absence of an event (or anomaly), the sound in installations after an event and the related events.

Such a sound may correspond to a bather diving, an object entering in the water installation, an object's presence in the installation's pipe, rain drops, bathers swimming or playing, air release under the water's surface, overflow problems, full skimmer baskets, different flow rate or an opening/closing of the cover, presence of an automatic cleaner operating.

This sound-based alert system may be used to detect a bather in distress and/or drowning.

In particular embodiments, such as the one shown in FIG. 9, the system 900 object of the present invention comprises an external parameter sensor 918, the trained model being configured to associate, for at least one series of sensed value representative of a physical/chemical parameter and at least one series of external parameter sensed, at least one aquatic installation operational degradation event associated with a date of event occurrence.

Such an external parameter sensor 918 may correspond to any physical/chemical sensor, such as a camera and/or a video camera taking picture and/or filming the installation 911, or digital sensor, such as a connector to a weather forecasting API configured to provide data representative of current or future weather in the proximity of the installation 911.

Such an external parameter sensor 918 may be configured to sense values representative of weather conditions, and/or air pollution, and/or a number of bathers in the aquatic installation, and/or air temperature, and/or water temperature, and/or movement detection, and/or face recognition, and/or a color change detection, and/or spots and/or stain detection in the installation, and/or size detection, and/or form acquisition and/or detection, and/or contrast detection within the installation's water for example.

The data representative of the values sensed by at least one sensor, 910, 915, 916, 917, 918, 981, 982, 983, 984 and/or 100, is transmitted to a computing system 1400, such as the one shown in FIG. 9.

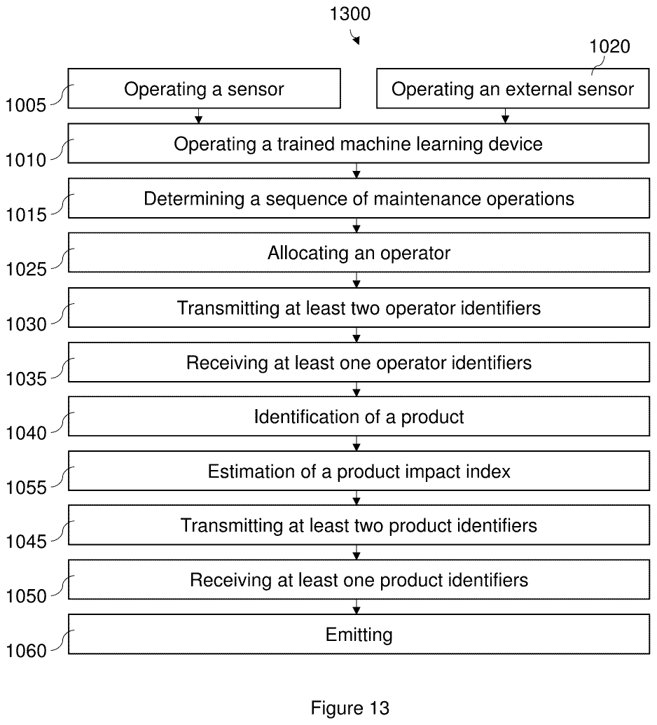

This computing system 1400 comprises at least one processor 920 configured to execute instructions, which can correspond to a computer software, representative of at least the following steps, such as shown as reference 1300 in FIG. 13:
operating 1010 a trained machine learning model, said model being trained to associate, for at least one series of sensed value representative of a physical/chemical parameter, at least one aquatic installation operational degradation event associated with a date of event occurrence, and
determining 1015 a sequence of maintenance operations to be performed on at least one aquatic installation as a function of at least one predicted aquatic installation operational degradation event and associated date of event occurrence.

During the step 1010 of operating, a trained machine learning model obtained during a step (not represented) of training said machine learning model is applied to data generated by operating at least one sensor 910, 915, 916, 917, 918, 981, 982, 983, 984 and/or 100.

The type of machine learning device used to obtain the trained machine learning model can be of any type suited for the nature and format of the data generated by operating at least one sensor 910, 915, 916, 917, 918, 981, 982, 983, 984 and/or 100.

Such a machine learning device can operate at least one of the following types of machine learning methods:
supervised learning, in which the machine learning model is trained to predict an output based on an input, given a set of examples,
unsupervised learning, in which the machine learning model is trained to identify patterns in the data without any guidance,
semi-supervised learning, in which the machine learning model is trained on both labeled and unlabeled data,
reinforcement learning, in which the machine learning model is trained based on the rewards and punishments it receives for its actions, and/or
deep learning, in which the machine learning model is trained using artificial neural networks to learn and make decisions.

Such a machine learning device can operate at least one of the following types of deep learning methods:
Convolutional Neural Networks (CNNs), often used for image and video processing tasks, such as object recognition, image classification, and segmentation,
Recurrent Neural Networks (RNNs), often used for sequential data processing, such as natural language processing (NLP), speech recognition, and time-series analysis, and/or
Attention Models, often used for machine learning models model needing to selectively focus on different parts of the input.

Such a machine learning device may use ensemble neural networks, for example.

The output of the step 1010 of operating is a list of at least one installation operational degradation event, that is an event requiring maintenance and preferably proactive maintenance, and dates of occurrence of said event.

Such an installation operational degradation event can correspond to, but is not limited to:
a low, critically low or empty level of a consumable used in a water treatment circuit associated with the installation 911, and/or
a device malfunction or breakdown, said device belonging to a water treatment circuit associated with the installation 911, or interacting with said installation 911, and/or
a state of the water in the installation 911, said state being representative of an acidity or basicity level, or a water behavior (scaling or aggressive) for example, and/or
a low or high or inappropriate disinfection rate and/or high or inappropriate disinfection residue rate (such as combined chlorine when active chlorine is used as the main disinfected agent) in the installation 911, said rate being measure with dedicated sensors, with values which are lower or higher to the user's set point and/or to the tolerate values by local regulations and/or by the disinfection algorithms which adjust continuously the ideal values based on the aquatic installation uses and real time need, and/or
a low or high or inappropriate value of a chemical parameter, such as total alkalinity and/or pH and/or cyanuric acid content, and/or salt content, and/or phosphates compounds rate, and/or sulfates rates, and/or nitrates rates, and/or nitrites rates, and/or chlorates rates, and/or
a low or high or inappropriate pressure measured in the filter and/or in the pipes and/or in the filtration pump, and/or
a low or high or inappropriate flow measured in the filter and/or in the pipes and/or in the filtration pump, and/or
a leak detection, and/or
an overuse of water refill, and/or
a low or high level of water in the installation, and/or
an overconsumption of energy, and/or
a high turbidity in the aquatic installation, and/or
a high amount of particle presence in the aquatic installation, and/or
a spot and/or stain detection, and/or
a high number of metallic compounds, and/or
the number of users and/or bathers at the aquatic facility, and/or
a predicted or unpredicted weather change that may occur in the coming period, and/or
the impact of short- and longer-term climactic changes on the aquatic facilities ability to function correctly and according to control parameters, and/or
a watercolor change, and/or
a detected presence of unallowed object and/or animal, and/or
a temperature raising failure or a non-homogenous temperature in the water installation.

The output date of the step 1010 of operating can be an absolute date (“March 3^{rd}”) or a relative date (“in a week”, “within 3 days”, “within 104 h”).

In more advanced embodiments, the step 1010 of operating is configured to associate other parameters to an installation operational degradation event occurrence prediction. Such other parameters may correspond to, for example, a level of criticality of the event or the operating time to solve it.

In more advanced embodiments, the step 1010 of operating is configured to associate several dates to an installation operational degradation event occurrence prediction. Such dates correspond to, for example, changes in criticality of the event such as, for example, the switch from a low level of a consumable to a critically low level or to an empty level.

In particular embodiments, the method 1300 object of the present invention further comprises a step 1020 of operating an external sensor, which can be of any type known to a person skilled in the art of aquatic facility monitoring, the step 1010 of operating a trained machine learning device being configured to operate as a function of a sensed parameter value originating from each said sensor.

The step 1015 of determining a sequence of maintenance operations to be performed corresponds, for example, to a resource allocation algorithm. Such a resource allocation algorithm may be configured to schedule, that is organize in time, a sequence of maintenance operations.

Such an organization may be based solely on the predicted dates of occurrence of events obtained during the step 1010 of operating.

In more advanced embodiments, such an organization may be based on secondary criteria, such as operator-event compatibility, product availability, geographical locations of the events and operating costs. Examples of such embodiments are disclosed below.

In particular embodiments, the at least one processor 920 is configured to execute instructions representative of a step 1025 of allocating, for at least one predicted event in a sequence of maintenance operations, an operator identifier as a function of operator parameters associated with the operator identifier.

In simple embodiments, during the step 1025 of allocating, each event in the sequence of events is associated to an event identifier and each operator is associated to an operator identifier.

An event identifier corresponds, for example, to a bijective code (such as an alphanumeric or binary code) which corresponds to the digital representation of an event. An event identifier may be associated to further information, such as the date of the event, a location of the event, a type of event and a status of the maintenance for the event, for example.

An operator identifier corresponds, for example, to a bijective code (such as an alphanumeric or binary code) which corresponds to the digital representation of an operator. An operator identifier may be associated to further information, such as the name of the operator, a type of events that the operator can process, a location of the operator and a status of the operator, for example.

This operator allocation can be automatic (entirely performed by a software), semi-automatic (based on suggestions by a software validated or overridden by a user) or manual (set by a user upon a graphical user interface for example).

The allocation may be materialized by the creation of a link (such as a key in a table in a database), in a memory, between an event identifier and an operator identifier.

Such an allocation may follow allocation rules, such as, for example:
an operator identifier cannot be associated to an event identifier if the operator identifier is associated to another event identifier at the same date and time or during an interval surrounding the associated event which cannot allow the operator to be totally available for the second event, and/or
an operator identifier cannot be associated to an event identifier if the event identifier is associated to a date that is below a determined threshold from the date of another event identifier already allocated to the operator that exceeds a determined threshold value from the location of said already allocated event.

In a multi-installation and multi-operator context, this means that a user and/or a software can allocate operators to the maintenance of installations on a many-to-many basis.

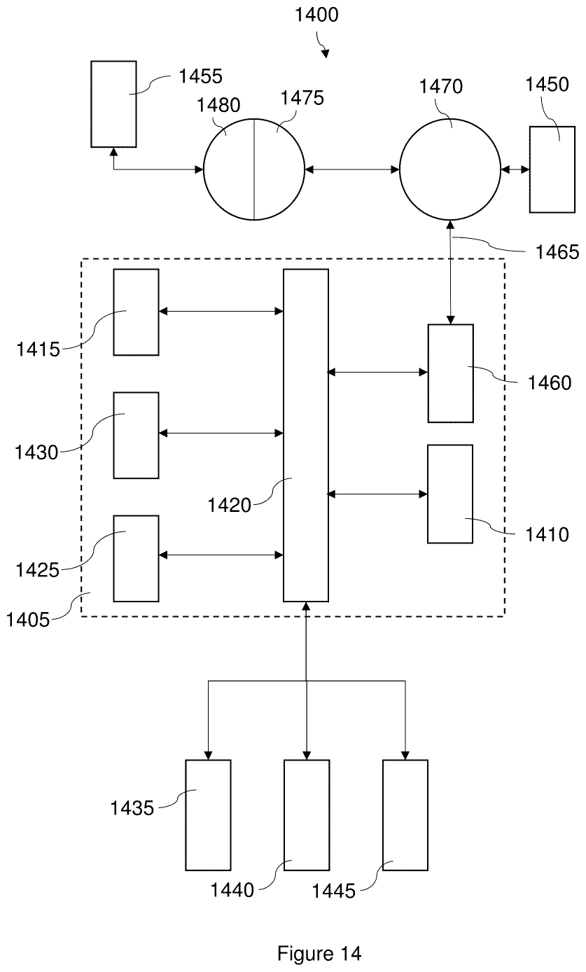

In particular embodiments, at least two operator identifiers are allocated during the step 1025 of allocation, at least one processor 920 being configured to execute instructions representative of the steps of:
transmitting 1030, to a third-party computing system associated with a user identifier, at least two said operator identifiers, and
receiving 1035, from a third-party computing system associated with the user identifier, a selection of at least one of the at least two said operator identifiers. The steps of transmitting 1030 and receiving 1035 may be performed using any communication means, or I/O subsystem such as shown in FIG. 14.

For example, during the step 1030 of transmitting, a digital message comprising at least two said operator identifiers allocated to a predicted event, as well the event identifier, is sent to a computing system belonging, or associated to, a user identifier. A user identifier corresponds, for example, to a bijective code (such as an alphanumeric or binary code) which corresponds to the digital representation of a user. A user identifier is typically associated to an aquatic facility owner or an aquatic facility manager in charge of several aquatic facilities.

The third-party computing system corresponds to, for example, a computer or smartphone belonging to the user. The message may also correspond to a notification, via email for example, inviting the user to connect upon a facility management software comprising a graphical user interface. Such a facility management software may allow for several functionalities:
aquatic installation status monitoring, and/or
aquatic installation maintenance event predictions, and/or
operator allocation for maintenance events, and/or
product purchase and allocation for maintenance events, and/or
self-help resources to autonomously performing maintenance upon water installation.

Using the third-party computing system, a user selects at least one operator identifier to handle the aquatic installation operational degradation event.

For example, during the step 1035 of receiving, a user input is registered upon a graphical user interface, said input being representative of a selection of at least one operator identifier. Such an input may be performed, for example, upon a smartphone or computer, triggering a selection of operator identifiers by the computing system 1400.

In particular embodiments, at least one predicted event is associated with an event type identifier, at least one operator parameter representing an event type identifier operator compatibility.

Such an event type identifier corresponds, for example, to a bijective code (such as an alphanumeric or binary code) which corresponds to the digital representation of a type of degradation event. Examples of such types of degradation events are mentioned above. Other such examples may correspond to, for example:
hydraulic circuit events, and/or
consumable refill events, and/or
installation maintenance events, and/or
electric repair maintenance events, and/or
filter related maintenance events, and/or
pump related maintenance events, and/or
installation equipment maintenance events, and/or
installation equipment sensor maintenance events.

Each predicted aquatic installation operational degradation event may be associated with at least one event type identifier and each operator may be associated with at least one event type identifier. This operator and event type association may be performed in a facility management software in which operators may log in and set up their account as well as event type associations. Such associations may also be performed by facility managers or swimming pool owners by registering operators and associating event types to said operators.

In particular embodiments, the at least one processor 920 is configured to execute instructions representative of a step 1040 of identification of at least one product identifier representative of a product to be used during the sequence of maintenance operations determined.

A product identifier corresponds, for example, to a bijective code (such as an alphanumeric or binary code) which corresponds to the digital representation of a product to be used in the context of the maintenance to be performed. A product identifier may correspond to a consumable or any aquatic installation equipment, for example.

The step 1040 of identification may be performed by reading, in a memory, product identifiers proactively associated to aquatic installation operational degradation event identifiers. Such an association can be performed automatically, semi-automatically or manually by a user.

Such identified products may be communicated to a user of the system, typically a user associated with the aquatic installation 911 subject of the maintenance.

In particular embodiments, at least two product identifiers are identified during the step 1040 of identification, at least one processor 920 being configured to execute instructions representative of the steps of:
transmitting 1045, to a third-party computing system associated with a user identifier, at least two said product identifiers, and
receiving 1050, from a third-party computing system associated with the user identifier, a selection of at least one of the at least two said product identifiers.

The steps of transmitting 1045 and receiving 1050 may function similarly to the steps of transmitting 1030 at least two operator identifiers and the step 1035 of receiving at least one operator identifier. Instead of selecting an operator, the user here selects a product.

In particular embodiments, the at least one processor 920 is configured to execute instructions representative of a step of estimation 1055 of a product impact index, representative of the capacity of a product to resolve an aquatic installation operational degradation event, said index being associated with at least one identified product identifier and transmitted during the step of transmitting.

Such a step of estimation 1055 may be performed, for example, by retrieving a product impact index from a product to event identifier matrix storing values representative of the impact of a particular product for a particular type of event. This product impact index is representative of the capacity of a particular product to qualitatively participate in resolving a particular event.

In particular embodiments, the at least one processor 920 is configured to execute instructions representative of a step of emitting 1060, to a third-party computing system associated with at least one selected product identifier, a message representative of a purchase order of at least one product associated with the at least one selected product identifier.

Such a step of emitting 1060 may be performed using an I/O subsystem or a communication means of a computing system 1400 such as shown in FIG. 14.

The message emitted may further comprise information representative of a shipping location for the purchased product.

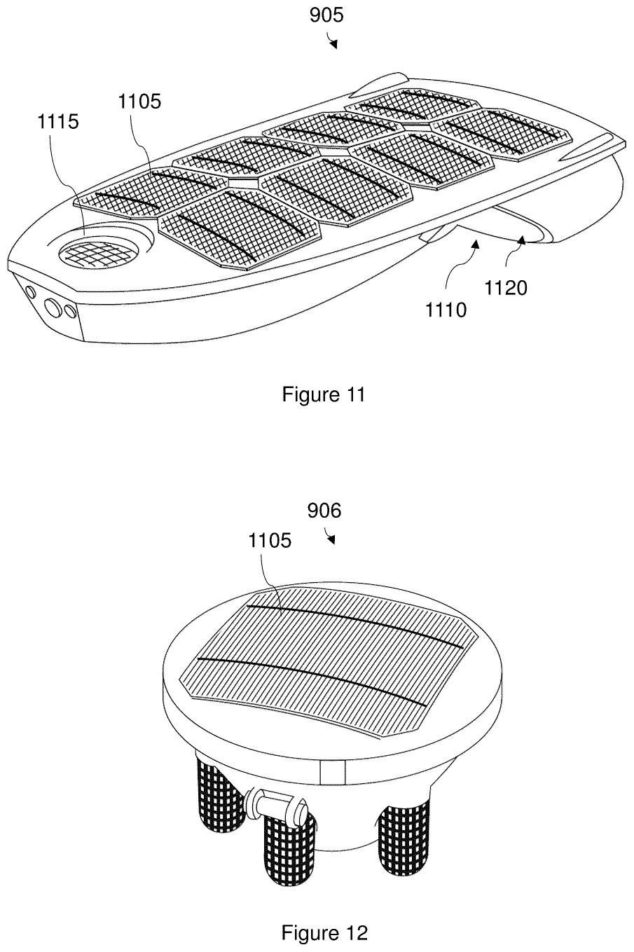

FIG. 10 represents, schematically, a particular succession of steps of the method 1000 object of the present invention. This aquatic installation predictive maintenance method 1000 comprises:
at least one step 1005 of operating a physical/chemical sensor interacting with water in at least one aquatic installation to provide series of at least one sensed value representative of a physical/chemical parameter,
a step 1010 of operating a trained machine learning model, said model being trained to associate, for at least one series of sensed value representative of a physical/chemical parameter, at least one aquatic installation operational degradation event associated with a date of event occurrence, and
a step 1015 of determining a sequence of maintenance operations to be performed on at least one aquatic installation as a function of at least one predicted aquatic installation operational degradation event and associated date of event occurrence.

Particular embodiments of the steps of operating physical/chemical sensor 1005, of operating 1010 a trained machine learning model and of determining 1015 are disclosed above.

FIG. 14 represents a block diagram that illustrates an example computer system 1400 with which an embodiment may be implemented. In the example of FIG. 14, a computer system 1405 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The computer system 1405 includes an input/output (IO) subsystem 1420 which may include a bus and/or other communication mechanism(s) for communicating information and/or instructions between the components of the computer system 1405 over electronic signal paths. The I/O subsystem 1420 may include an I/O controller, a memory controller and at least one I/O port. The electronic signal paths are represented schematically in the drawings, for example as lines, unidirectional arrows, or bidirectional arrows.

At least one hardware processor 1410 is coupled to the I/O subsystem 1420 for processing information and instructions. Hardware processor 1410 may include, for example, a general-purpose microprocessor or microcontroller and/or a special-purpose microprocessor such as an embedded system or a graphics processing unit (GPU) or a digital signal processor or ARM processor. Processor 1410 may comprise an integrated arithmetic logic unit (ALU) or may be coupled to a separate ALU.

Computer system 1405 includes one or more units of memory 1425, such as a main memory, which is coupled to I/O subsystem 1420 for electronically digitally storing data and instructions to be executed by processor 1410. Memory 1425 may include volatile memory such as various forms of random-access memory (RAM) or other dynamic storage device. Memory 1425 also may be used for storing temporary variables or other intermediate information during execution of instructions to be executed by processor 1410. Such instructions, when stored in non-transitory computer-readable storage media accessible to processor 1410, can render computer system 1405 into a special-purpose machine that is customized to perform the operations specified in the instructions.

Computer system 1405 further includes non-volatile memory such as read only memory (ROM) 1430 or other static storage device coupled to the I/O subsystem 1420 for storing information and instructions for processor 1410. The ROM 1430 may include various forms of programmable ROM (PROM) such as erasable PROM (EPROM) or electrically erasable PROM (EEPROM). A unit of persistent storage 1415 may include various forms of non-volatile RAM (NVRAM), such as FLASH memory, or solid-state storage, magnetic disk, or optical disk such as CD-ROM or DVD-ROM and may be coupled to I/O subsystem 1420 for storing information and instructions. Storage 1415 is an example of a non-transitory computer-readable medium that may be used to store instructions and data which when executed by the processor 1410 cause performing computer-implemented methods to execute the techniques herein.

The instructions in memory 1425, ROM 1430 or storage 1415 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, 14ML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 1405 may be coupled via I/O subsystem 1420 to at least one output device 1435. In one embodiment, output device 1435 is a digital computer display or Human Machine Interface. Examples of a display that may be used in various embodiments include a touch screen display or a light-emitting diode (LED) display or a liquid crystal display (LCD) or an e-paper display. Computer system 1405 may include other type(s) of output devices 1435, alternatively or in addition to a display device. Examples of other output devices 1435 include printers, ticket printers, plotters, projectors, sound cards or video cards, speakers, buzzers or piezoelectric devices or other audible devices, lamps or LED or LCD indicators, haptic devices, actuators, or servos.

At least one input device 1440 is coupled to I/O subsystem 1420 for communicating signals, data, command selections or gestures to processor 1410. Examples of input devices 1440 include touch screens, microphones, still and video digital cameras, alphanumeric and other keys, keypads, keyboards, graphics tablets, image scanners, joysticks, clocks, switches, buttons, dials, slides.

Another type of input device is a control device 1445, which may perform cursor control or other automated control functions such as navigation in a graphical interface on a display screen, alternatively or in addition to input functions. Control device 1445 may be a touchpad, a mouse, a trackball, or cursor direction keys for communicating direction information and command selections to processor 1410 and for controlling cursor movement on display 1435. The input device may have at least two degrees of freedom in two axes, a first axis (e.g., 14) and a second axis (e.g., y), that allows the device to specify positions in a plane. Another type of input device is a wired, wireless, or optical control device such as a joystick, wand, console, steering wheel, pedal, gearshift mechanism or other type of control device. An input device 1440 may include a combination of multiple different input devices, such as a video camera and a depth sensor.

In another embodiment, computer system 1405 may comprise an internet of things (IoT) device in which one or more of the output device 1435, input device 1440, and control device 1445 are omitted. Or, in such an embodiment, the input device 1440 may comprise one or more cameras, motion detectors, thermometers, microphones, seismic detectors, other sensors or detectors, measurement devices or encoders and the output device 1435 may comprise a special-purpose display such as a single-line LED or LCD display, one or more indicators, a display panel, a meter, a valve, a solenoid, an actuator or a servo.

Computer system 1405 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 1405 in response to processor 1410 executing at least one sequence of at least one instruction contained in main memory 1425. Such instructions may be read into main memory 1425 from another storage medium, such as storage 1415. Execution of the sequences of instructions contained in main memory 1425 causes processor 1410 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage 1415. Volatile media includes dynamic memory, such as memory 1425. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 1420. Transmission media can also take the form of acoustic or light waves, such as those generated during radio-wave and infra-red data communications.

Various forms of media may be involved in carrying at least one sequence of at least one instruction to processor 1410 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 1405 can receive the data on the communication link and convert the data to a format that can be read by computer system 1405. For instance, a receiver such as a radio frequency antenna or an infrared detector can receive the data carried in a wireless or optical signal and appropriate circuitry can provide the data to I/O subsystem 1420 such as place the data on a bus. I/O subsystem 1420 carries the data to memory 1425, from which processor 1410 retrieves and executes the instructions. The instructions received by memory 1425 may optionally be stored on storage 1415 either before or after execution by processor 1410.

Computer system 1405 also includes a communication interface 1460 coupled to bus 1420. Communication interface 1460 provides a two-way data communication coupling to network link(s) 1465 that are directly or indirectly connected to at least one communication network, such as a network 1470 or a public or private cloud on the Internet. For example, communication interface 1460 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 1470 broadly represents a local area network (LAN), wide-area network (WAN), campus network, internetwork, or any combination thereof. Communication interface 1460 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 1460 sends and receives electrical, electromagnetic, or optical signals over signal paths that carry digital data streams representing various types of information.

Network link 1465 typically provides electrical, electromagnetic, or optical data communication directly or through at least one network to other data devices, using, for example, satellite, cellular, Wi-Fi, or BLUETOOTH technology. For example, network link 1465 may provide a connection through a network 1470 to a host computer 1450.

Furthermore, network link 1465 may provide a connection through network 1470 or to other computing devices via internetworking devices and/or computers that are operated by an Internet Service Provider (ISP) 1475. ISP 1475 provides data communication services through a world-wide packet data communication network represented as internet 1480. A server computer 1455 may be coupled to internet 1480. Server 1455 broadly represents any computer, data center, virtual machine, or virtual computing instance with or without a hypervisor, or computer executing a containerized program system such as DOCKER or KUBERNETES. Server 1455 may represent an electronic digital service that is implemented using more than one computer or instance and that is accessed and used by transmitting web services requests, uniform resource locator (URL) strings with parameters in HTTP payloads, API calls, app services calls, or other service calls. Computer system 1405 and server 1455 may form elements of a distributed computing system that includes other computers, a processing cluster, server farm or other organization of computers that cooperate to perform tasks or execute applications or services. Server 1455 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, 14ML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 1455 may comprise a web application server that hosts a presentation layer, application layer and data storage layer such as a relational database system using structured query language (SQL) or no SQL, an object store, a graph database, a flat file system or other data storage.

Computer system 1405 can send messages and receive data and instructions, including program code, through the network(s), network link 1465 and communication interface 1460. In the Internet example, a server 1455 might transmit a requested code for an application program through Internet 1480, ISP 1475, local network 1470 and communication interface 1460. The received code may be executed by processor 1410 as it is received, and/or stored in storage 1415, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 1410. While each processor 1410 or core of the processor executes a single task at a time, computer system 1405 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

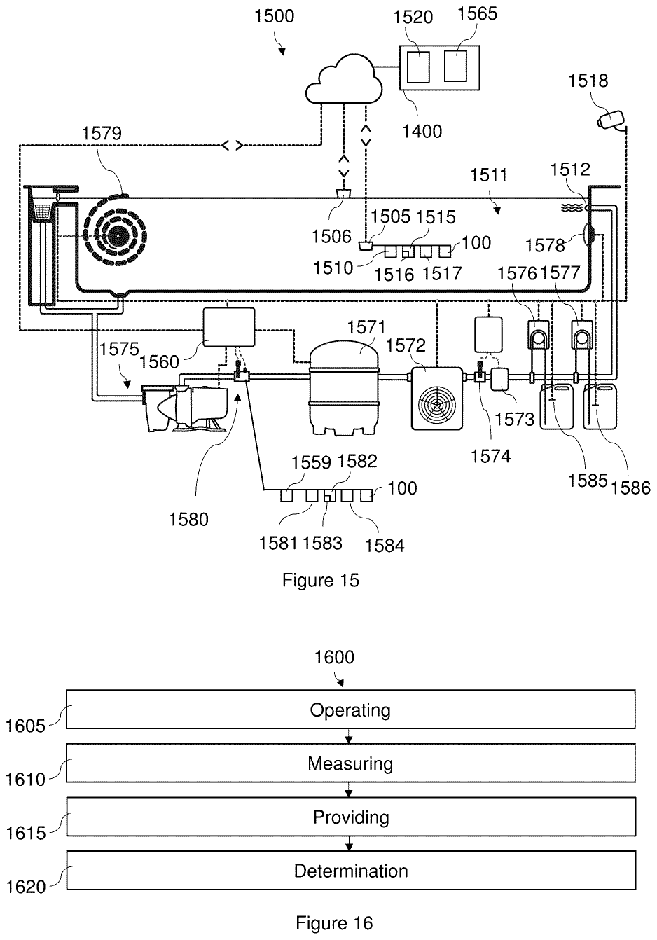

FIG. 15 represents, schematically, a particular embodiment of the system 1500 object of the present invention. This aquatic installation monitoring system 1500, characterized in that it comprises:
at least one physical/chemical sensor, 1510, 1517, 1559, 1581, 1582, 1583 and/or 1584, configured to provide series of at least one sensed value representative of a local physical/chemical parameter,
at least one optical sensor, 1515, 1516 and/or 1518, and
an aquatic installation state determination means 1520, comprising a computing device configured to receive and to process at least one series of a local physical/chemical parameter and/or at least one graphical representation to determine a value representative of an aquatic installation state.

In particular embodiments, the system 1500 comprises a submersible and/or floating vehicle, 1505 and/or 1506, comprising:
at least one said physical/chemical sensor, 1510 and/or 1517, configured to provide series of at least one sensed value representative of a local physical/chemical parameter, and
at least one said optical sensor, 1515 and/or 1516, configured to provide a graphical representation of water in the aquatic installation and/or the aquatic installation.

In particular embodiments, the system 1500 comprises at least one external sensor 1518, configured to provide an external graphical representation and/or video and/or picture acquisition of water in the aquatic installation and/or the aquatic installation and/or to provide external factors and/or parameters acquisition.

The submersible and/or floating vehicle, 1505 and/or 1506, may correspond, for example, to any manually, remotely and/or automatically dirigible vehicle adapted to the particular use case.

The vehicle 1505 may correspond to a remotely or autonomously dirigible submarine vehicle, for example, such as shown in FIG. 11.

The vehicle 1506 may also correspond to a floating pod, such as shown in FIG. 12.

In particular embodiments, such as the one shown in FIG. 15, the system 1500 comprises both a submersible vehicle (ROV) 1505 and a floating vehicle 1506.

In particular variants, at least one submersible and/or floating vehicle, 1505 and/or 1506, comprises a solar panel 1105 configured to power an autonomous electricity source (not represented) and/or to charge the onboarded batteries (not represented).

In particular variants, at least one submersible and/or floating vehicle, 1505 and/or 1506, comprises an induction current collector configured to power an autonomous electricity source (not represented).

In particular variants, at least one submersible and/or floating vehicle, 1505 and/or 1506, comprises a power inlet configured to be connected to a charging wire to power an autonomous electricity source (not represented).

In particular variants, at least one submersible and/or floating vehicle, 1505 and/or 1506, comprises a propulsion 1110 system, such as an engine associated with a boat propeller. Such a propulsion 1110 system allows for the vehicle, 1505 and/or 1506, to move around in the water in the aquatic installation 1511. This propulsion system 1110 may comprise rear propellers 1120, configured to generate forward, backward or yaw movements, and a front propeller 315, configured to generate upward or downward movements.

Such a submersible and/or floating vehicle, 1505 and/or 1506, may further comprise a relative positioning coordinates acquisition means 1510, configured to locate, in a three-dimensional space representative of an aquatic installation 1511, the submersible vehicle, 1505 and/or 1506, and to provide the corresponding coordinates of the submersible and/or floating vehicle, 1505 and/or 1506.

Such a relative positioning coordinates acquisition means 1510 is, for example, a sonar configured to provide distance values from edges of the aquatic installation 1511. The distance values allow for the determination of the shape of the aquatic installation 1511. Once the shape of the aquatic installation 1511 is known, such distance values allow for the determination of the positioning of the submersible and/or floating vehicle, 1505 and/or 1506, within said aquatic installation 1511.

In another variant, the relative positioning coordinates acquisition means 1510 is, for example, a mechanical sensor used in coordination with a propulsion system 1110 to map the shape of the aquatic installation 1511 by detecting collisions of the submersible and/or floating vehicle, 1505 and/or 1506, with the edges of this aquatic installation 1511.

In another variant, the relative positioning coordinates acquisition means 1510 is, for example, detected by the external sensor 1518, to map the shape of the aquatic installation 1511 by detecting the edges of this aquatic installation 1511 and the position of the submersible and/or floating vehicle, 1505 and/or 1506. In such a variant, the submersible and/or floating vehicle, 1505 and/or 1506 may be linked to the external sensor 1518 to avoid any collision with the edge of this aquatic installation 1511.

Once the shape of the aquatic installation 1511 is known, information originating from original parameters of the propulsion system 1110 may be used to locate the submersible and/or floating vehicle, 1505 and/or 1506. For example, a duration of use of the propulsion system 1110, associated with a power of propulsion, may be used in a calculation to determine a distance of the submersible and/or floating vehicle, 1505 and/or 1506, from the last known location.

The data resulting from the physical and/or chemical sensing means 1515 may further be associated to a spatial value obtained by a timestamping means configured to associate a value representative of a time of capture to the data resulting from the physical and/or chemical sensing means 1515 to form a series of timestamped local physical and/or chemical value sensed.

The data resulting from the physical and/or chemical sensing means 1515 may further be associated to a spatial value obtained by the relative positioning coordinates acquisition means 1510 to form a series of local physical and/or chemical value sensed. This data may further be associated with environmental context values, such as pressure or time of capture for example.

The combination of the use of a timestamping means and a relative positioning coordinates acquisition means allows the formation of a series of local timestamped physical and/or chemical value sensed.

In particular variants, the submersible and/or floating vehicle, 1505 and/or 1506, comprises an aquatic installation local physical and/or chemical state information aggregation means.

Data originating from different sensors, timestamping means or relative positioning coordinates acquisition means may be processed by an aquatic installation local physical and/or chemical state information aggregation means. Such an aggregation means is, for example, a computer software executed upon a computing device 1400, such as the one shown in FIG. 14. This computing device 1400 is configured to associate, in a memory, the data resulting from the relative positioning coordinates acquisition means 1510, the physical and/or chemical sensor, 1510 and/or 1517, and/or an optical sensor, 1515 and/or 1516, and/or a timestamping means.

Such an association may be performed by concatenating said data in a single data stream or data frame or by creating a link between said data if such data is stored in separate database tables for example.

The timestamping means may correspond, for example, to any electronic clock used by a computing device. Such a timestamping means may be integrated into the submersible and/or floating vehicle, 1505 and/or 1506, or be remotely located from said submersible and/or floating vehicle, 1505 and/or 1506. By remotely located, it is intended that the timestamping means is linked with the submersible and/or floating vehicle, 1505 and/or 1506, by a communication means, such as a peer-to-peer link or a communications network link, such as the Internet for example.

The system 1500 further comprises a water physical and/or chemical sensor 1510. Such a water physical and/or chemical sensor 1510 may be associated with a submersible and/or floating vehicle, 1505 and/or 1506.

Such a water physical and/or chemical sensor 1510 is intended in the broadest sense, meaning that any physical and/or chemical parameter sensing device is encompassed, provided the output data of such a device is used to evaluate the physical and/or chemical state of the water in the aquatic installation 1511.

The data provided by the physical/chemical sensor, 1510 and/or 1517, and the optical sensor, 1515 and/or 1516, may also be complemented by data originating from at least one further physical and/or chemical sensor, 1559, 1581, 1582, 1583 and/or 1584, is situated in an analysis chamber 1580, or in a pipe of a circulation system where water flows, interacting with water in at least one aquatic installation and configured to provide series of at least one sensed value representative of a physical/chemical parameter. Such a physical/chemical parameter may be representative of a state of operation of the aquatic installation and/or of the water in the aquatic installation 1511.

The physical and/or chemical sensor, 1510, 1515, 1516, 1517, 1559, 1581, 1582, 1583 and/or 1584, is intended in the broadest sense, meaning that any physical and/or chemical parameter sensing device is encompassed, provided the output data of such a device is used to evaluate the physical and/or chemical state of the water in an aquatic installation 1511 and/or the physical and/or chemical state of the aquatic installation 1511, and/or status of equipment in the aquatic installation.

Such a physical and/or chemical sensor, 1510, 1515, 1516, 1517, 1559, 1581, 1582, 1583 and/or 1584, may correspond to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
an optical sensor, and/or
a camera and/or video camera, and/or
an infrared sensor, and/or
an acoustic and/or sonar sensor, and/or
a temperature sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

The submersible and/or floating vehicle, 1505 and/or 1506, further comprises an optical sensor 1515. Such an optical sensor 1515 corresponds, for example, to a camera or to a video camera configured to capture images of the aquatic installation 1511 and/or the water in the aquatic installation 1511.

In particular embodiments, such as the one represented in FIG. 15, the optical sensor 1515 comprises an infrared sensor 1516.

The system 1500 further comprises an aquatic installation state determination means 1520. Such an aquatic installation state determination means 1520 corresponds to a computing device. Such a computing device is, for example, configured to execute instructions corresponding to a computer software. An example of such a computing device 1400, or computer system, is shown in regard to FIG. 14.

This aquatic installation state determination means 1520 may be, for example, a computer software executed upon a computing device. This aquatic installation parameter determination means 1520 may use an algorithmic module or a machine learning module to link a parameter value to at least one value sensed by the submersible and/or floating vehicle, 1505 and/or 1506, and/or by any other sensor associated with the aquatic installation 1511 and/or by any other sensor associated with a hydraulic circuit 1575 associated with the aquatic installation 1511.

An algorithmic module comprises a series of mathematical operations to be performed on a set or stream of data whereas a machine learning module comprises a machine learning architecture used on a training set or stream of data in order to produce a trained machine learning model that may then be used with operational data.

The aquatic installation state determination means 1520 can be associated with at least one communication interface such as shown in regard to FIG. 14.

The aquatic installation state determination means 1520 is configured to determine a value representative of a water and/or aquatic installation state as a function of data emitted by a physical/chemical sensor 1510 and/or an optical sensor 1515. The nature of the state determined is variable and dependent on the particular use case of the system 1500.

In particular embodiments, such as the one represented in FIG. 15, the submersible and/or floating vehicle, 1505 and/or 1506, comprises a sonar sensor 1517 configured to provide an output, the aquatic installation state determination means 1520 being configured to determine an aquatic installation state as a function of the output provided by the sonar sensor.

Based on the sound modifications perceived by the sonar sensor, notifications and/or alerts can be emitted.

For example, a pump which starts operating provides a specific sound (vibrating signature), these vibrations follow the water flow into the aquatic installation 1511. Therefore, the use of a sonar sensor allows for the measurement and analysis of these vibrations. Any change in this signature thus results from an interaction and/or a problem which has occurred between the pump and the aquatic installation 1511. Such an interaction could typically correspond to a non-activation of the pump, a pump surge, a pump cavitation, an object/pollution in the pipe, a leak or a product injection. By measuring a difference between the normal state sound and the current measured sound, the diagnostic can be made. This difference can also be linked to other types of data collected.

Any sound that may occur in the aquatic installation 1511 can be linked to a type, or class, of event. The determination of this class can be obtained by using a trained machine learning classifier model. Such a trained machine learning classifier model can be obtained by feeding, in a machine learning classifier device, a sample comprising sounds and the related events. Such a trained machine learning classifier model can be obtained by feeding, in a machine learning classifier device, a sample comprising the sound in installations in the absence of an event (or anomaly), the sound in installations after an event and the related events.

Such a sound can correspond to a bather diving, an object entering in the water installation, rain drops, bathers swimming or playing, air release under water by bathers, overflow problems, full skimmer baskets, different flow rate or an opening/closing of the cover.

This sound-based alert system may be used to detect a bather drowning.

In particular embodiments, the system 1500 object of the present invention comprises a water physical/chemical treatment device, 1572, 1573, 1576 and/or 1577, configured to modify a water physical and/or chemical parameter as a function of the aquatic installation state determined.

Such a water physical and/or chemical treatment device may correspond to any actuator 1560 suited for the particular use case of the system 1500 object of the present invention. Such an actuator 1560 may be one regularly used within or in combination of a hydraulic circuit or aquatic installation.

Another element may correspond to a heat pump 1572 associated to an actuator (160) configured to increase or decrease the temperature of the water. Such an actuator 1560 may be linked to a remote computing device 1565.

Another element may correspond to a disinfectant device 1573 (electro-chlorinator system, ultraviolet system, or ozone generator system or any possible in situ disinfectant generator or any solid disinfectant feeder which dissolution and injection could be controlled by an actuator) associated to an actuator (160) configured to increase or decrease the activation of the disinfectant device 1573. Such an actuator 1560 may be linked to a remote computing device 1565.

Such a disinfectant device 1573 may further be associated with an analysis chamber 1574 associated with at least one sensor (not referenced). Such a sensor may be configured to detect a level of bacterial and/or algae activity and/or a disinfectant adjustment need in a sample of water from the aquatic installation 1511. Such a sensor may also be configured to measure free chlorine and/or total chlorine to determine combined chlorine and manage the chlorine need and adjust the electrochlorination process. Such a sensor may also be configured to detect a flow or an absence of flow in the electrochlorinator cell as a safety additional equipment to avoid any electrochlorination process with absence of flow.

Another element may correspond to a pH adjustment device 1576 associated to an actuator 1560 configured to increase or decrease the pH of the water. Such an actuator 1560 may be linked to a remote computing device 1565.

Such a pH adjustment device 1576 may be associated to a pH sensor (not represented) and/or a pH adjustment chemical compound drum level 1585.

Another element may correspond to a disinfectant (such as liquid chlorine or sodium hypochlorite or any liquid disinfectant) release device 1577 associated to an actuator 1560 configured to increase or decrease the concentration of disinfectant in the water. Such an actuator 1560 may be linked to a remote computing device 1565.

Such a disinfectant release device 1577 may be associated to a disinfectant chemical compound drum level 1586.

Such a disinfectant release device 1577 may further be associated with an analysis chamber 1580 associated with at least one sensor (159, 1581, 1582, 1583 and/or 1584). Such a sensor may be configured to detect a level of bacterial activity and/or a disinfectant adjustment need in a sample of water from the aquatic installation 1511. Such a sensor may also be configured to measure the disinfectant rate and/or the free chlorine and/or total chlorine to determine combined chlorine and/or the disinfectant residuals and manage the chlorine and/or disinfectant need and adjust the disinfectant release process. Such a sensor may also be configured to detect a flow or an absence of flow in the pipe and/or in the analysis chamber as a safety additional equipment to avoid any disinfectant release process with absence of flow.

Another element may correspond to an algicide rate adjustment device (not referenced) associated to an actuator 1560 configured to increase the algicide rate in the water base. Such an actuator 1560 may be linked to a remote computing device 1565.

Such an algicide rate adjustment device (not referenced) may be associated to a bacterium and/or algae activity sensor (located in an analysis chamber 1580) or an algicide rate adjustment chemical compound drum level. Such a sensor may be configured to detect a level of bacterial activity in a sample of water from the installation 1511.

Another element may correspond to a flocculant and/or clarifier adjustment device (not referenced) associated to an actuator 1560 configured to decrease the turbidity in the water base. Such an actuator 1560 may be linked to a remote computing device 1565.

Such a flocculant and/or clarifier adjustment device (not referenced) may be associated to a turbidity sensor (located in an analysis chamber 1580) or a flocculant and/or clarifier adjustment chemical compound drum level. Such a sensor may be configured to detect a level of turbidity in a sample of water from the installation 1511.

Another element may correspond to a liquid additive treatment adjustment device (not referenced) associated to an actuator 1560 configured to improve the water treatment. Such an actuator 1560 may be linked to a remote computing device 1565.

Such a liquid additive treatment adjustment device (not referenced) may be associated to a specific sensor (located in an analysis chamber 1580) or a liquid additive drum level (not referenced). Such a sensor may be configured to detect the need for an additive treatment injection in a sample of water from the installation 1511.

Another element may correspond to a light 1578 configured to illuminate the water in the aquatic installation 1511 and associated to an actuator (not referenced) configured to activate or deactivate the light 1578. Such an actuator 1560 may be linked to a remote computing device 1565. The activation of such a light 1578 increases the performance of image-based sensors, such as particle sensors and/or turbidity/clarity sensors.

Another element may correspond to an installation cover 1579 configured to selectively cover the aquatic installation 1511.

This installation cover 1579 may be associated with an actuator 1560 configured to open or close the cover 1579, for purposes of safety, water pollution reduction, evaporation control, and/or energy cost reduction for example. Such an actuator 1560 may be linked to a remote computing device 1565.

This installation cover 1579 may be associated with a sensor (not referenced) and/or with an external sensor 1518 configured to monitor the position of the cover 1579.

In particular embodiments, the system 1500 object of the present invention comprises a command emitter configured to emit a command representative of a target operational value for an actuator 1560 interacting with the physical and/or chemical state of the aquatic installation.

The command emitter is, for example, a computer software executed upon a computing device and associated to a communication means linking the emitter to the actuator 1560.

This command emitter is, for example, activated as a function of the result of a comparison between the value of a parameter sensed by the submersible and/or floating vehicle, 1505 and/or 1506, and a predetermined target value. Such a target value corresponds to, for example, a value representative of a desired physical and/or chemical state of the water in the aquatic installation 1511.

Such a command may correspond to, but is not limited to, for example:
a release or the end of a release of a chemical compound in the water of the aquatic installation 1511, and/or
an increase or a decrease in a water pump activation, and/or
an increase or a decrease in a water pump flow, and/or
an increase or a decrease in a water heater activation.

In particular embodiments, the water physical/chemical sensor 1510 and/or the external sensor 1518 is configured to measure a flow intensity from an inlet 1512 in the installation, the aquatic installation state determination means 1520 being configured to determine a pump flow efficiency as a function of the flow intensity.

The higher the flow is, the greater the travelled distance by the water pushed out of the inlet is. This also correlates with a higher water movement at the surface.

In particular embodiments, the water movement at the surface is measured by an external sensor 1518 and correlated to the water flow through the actuator 1560.

Therefore, the measurement of the intensity of such a flow allows the determination of an anomaly in a hydraulic circuit associated with the inlet 1512. Such an intensity may be measured in an initial state of the hydraulic circuit, corresponding to a nominal operating mode, by positioning the submersible and/or floating vehicle, 1505 and/or 1506, at a particular distance of the inlet, and/or by the distance of the water movement from the inlet measured by an external sensor 1518. Then, this intensity can be measured in the same location regularly, an anomaly being detected when the intensity measured is substantially different from the nominal intensity.

Based on the loss of the flow intensity out of the inlet, the state determination means 1520 can detect the loss of pump flow efficacy and diagnose the cause in correlation with the filter pressure and pump speed.

Below, several examples of diagnosis are provided:
Example 1: for the same filter pressure and pump speed, a loss of flow intensity can be generated by a leak in the pipes after the filter 1571.
Example 2: for the same pump speed, an increase of pressure in the filter 1571 and a loss of flow intensity are linked to a saturated filter 1571 and generate an alert and/or can trigger the activation of a filter cleaning.
Example 3: a higher pump speed increases the flow intensity. If not, the state determination means 1520 detects if it is a leak (same filter pressure) or a saturated filter (increase of the filter pressure).

In particular embodiments, the aquatic installation state determination means 1520 is configured to determine a water clarity value as a function of a graphical representation provided.

Such a water clarity value may be determined as a function of the distance required for the optical sensor of the submersible and/or floating vehicle, 1505 and/or 1506, to detect a determined or predetermined target. Such a target may correspond, for example, to a water inlet of an aquatic installation 1511. The definition of such a target may be initiated by an operator, by positioning the submersible and/or floating vehicle, 1505 and/or 1506, in a particular location of the aquatic installation 1511, commanding the capture of an image by the optical sensor and storing the coordinates of the vehicle at the moment of capture. These coordinates allow later image captures in the same coordinates, closer to the target or farther from the target.

Below, several examples of diagnosis are provided:
Example 15: low point acquisition (turbid water) and a high pressure in the filter 1571 of a hydraulic circuit associated with the aquatic installation 1511 may trigger a filter cleaning command and/or emission of an alert.
Example 2: low point acquisition (turbid water) and an adequate filter pressure can trigger the increase of the pump speed (or filtration time) if chemical state values are not good enough in the aquatic installation 1511.

In particular embodiments, the aquatic installation state determination means 1520 is configured to determine a value representative of the presence of particles in the water as a function of a graphical representation provided.

Particles may be detected, for example, during the night, by switching on lights 1578 aiming at the aquatic installation 1511 and capturing images of the reflection of particles inside the water in the installation using an external sensor 1518 and/or an optical sensor 1515. These reflections can be counted to give an average of the particles inside the water.

Based on this value, a pump speed and filtration time can be adjusted to allow the deposition of those particles inside a filter 1571 (the lowest the speed is, the easiest the particles are kept in the filter) and/or the use of a coagulant and/or flocculant product can be piloted to help the lower the number of particles inside the filter 1571.

In particular embodiments, the aquatic installation state determination means 1520 is configured to determine a value representative of the presence of the nature of an impurity in the water as a function of a graphical representation provided using an external sensor 1518 and/or an optical sensor 1515.

Such an impurity may be detected via an image processing algorithm.

When such an impurity is detected, as a new color or form detection on a coating of the aquatic installation 1511, an algorithm may determine the nature of the spot based on its form and color.

Below, examples of such detections are provided:
Example 15: In the case of leaves, dark spots which move with the flow can be detected, and an aquatic installation 1511 cleaner can be activated to remove said leaves.
Example 2: In the case of algae spot detection (green spots), an aquatic installation 1511 cleaner and a disinfection boost correlated with a filtration time and pump speed increase are activated, and/or an aquatic installation 1511 cleaner can be activated to brush said algae.
Example 3: In the case of mushrooms detection (pink spots), or rust (brown spot) an alert can be provided, this alert further provided instructions detailing how to remove said mushrooms or rust.

In particular embodiments, the aquatic installation state determination means 1520 is configured to determine a value representative of the presence of an animal in the water as a function of a graphical representation provided.

Such a presence may be detected by an image processing algorithm configured to recognize shapes of animals or humans, for example. In other variants, the amount of noise generated by turbulences in the water associated with the motions of the animal or human may trigger the detection of this presence.

In particular embodiments, the aquatic installation state determination means 1520 is configured to determine a value representative of a movement pattern of the animal in the water as a function of a graphical representation provided.

Such a presence may be detected by an image processing algorithm configured to recognize types or speed of movements of animals or humans, for example. In other variants, the amount of noise generated by turbulences in the water associated with the types of movements of the animal or human may trigger the detection of this presence.

It is possible to secure access to an aquatic installation 1511 by registering authorized persons in an area around the pool. Such persons may be associated with a picture, for example, and recognized by the optical sensor 1515 and/or 1518.

Moreover, the detection of a human size detection further allows the prevention of children drowning by emitting an alert in case of children presence close to the aquatic installation 1511.

By analyzing the body movement, it is possible to differentiate a bather swimming from a bather drowning.

This system can also measure the number of bathers at the same time in the aquatic installation 1511, in order to adjust the water treatment consequently, based on the number of bathers, their size and activities, to neutralize the caused pollution by disinfectant injection and pH adjustment.

In particular embodiments, the aquatic installation state determination means 1520 is configured to determine the temperature of the water based upon the output of an infrared sensor 1516 of the optical sensor 1515 and/or 1518.

The use of such an infrared capture provides the temperature flow in the water and at its surface. Correlated to an algorithm, the temperature value of the water can be determined.

Such an information may be used in the following scenarios:
Example 15: a non-homogenous temperature detected at the pool surface can trigger a command to the pump to increase its speed or its filtration time to favorize the uniformity of the temperature.
Example 2: an optimum filtration time and pump speed, and a non-homogeneous temperature detected, can trigger a command to increase the heat pump working time and/or will close the cover if any.
Example 3: the infrared capture can determine the evaporation of water in real time and close the cover (if any) to prevent water, total alkalinity and temperatures losses by evaporation, and/or reduce the heat pump work to lower the water temperature to reduce these losses.

In particular embodiments, the system 1500 further comprises a remote computing device configured to control, register and adjust a set of predetermined aquatic installation 1511 operational parameters values (such as the pH or total alkalinity for example).

The remote computing device may be, for example, operated as such:
All the data is routed by a traffic manager to a dedicated cloud (computing device and/or computer software):
   in each computing device and/or computing software, an algorithm manages the data's individual value from the camera acquisition of at least one vehicle, 1505 and/or 1506, and/or acquired by the external sensor 1518, and
   in a second step, an algorithm manages a combined global data value, and sends this data to a big data management module using the traffic manager.
The global data from at least one vehicle, 1505 and/or 1506, and an external sensor 1518 are compared to a user-set point:
   in case of differences between these values, the aquatic installation 1511 operational parameters are adjusted using water treatment and equipment piloting improvements,
   in parallel, a timestamped three-dimensional mapping of the aquatic installation 1511 is recorded, and risk-determining algorithms are executed to determine the presence and severity of risks in the water aquatic installation 1511 a plurality of zones, and
   a timestamped three-dimensional map is generated comprising a color code for the pool risks to identify the risk prone zones in a simplified manner.

In particular embodiments, the system 1500 comprises a total alkalinity measurement device, such as shown in FIGS. 1 and 2.

FIG. 16 represents, schematically, a particular succession of steps of the method 200 object of the present invention. This aquatic installation monitoring method 200 comprises:
a step 1605 of operating:
   at least one water physical/chemical sensor, and
   at least one optical sensor.
a step 1610 of measuring a local physical/chemical state of the water environment in the proximity of a submersible and/or floating vehicle and/or of measuring at least one external factor and/or parameter using the sensor 1518,
a step 1615 of providing a sensed graphical representation of the water and/or aquatic installation, and
an aquatic installation state determination step 1620, comprising a computing device configured to receive and to process measures of a local physical/chemical state of the water and/or graphical representations of the water to determine a value representative of an aquatic installation state and/or of its environment.

Particular implementations of the method 1600 object of the present invention are disclosed in relation to the system 1500 object of the present invention.

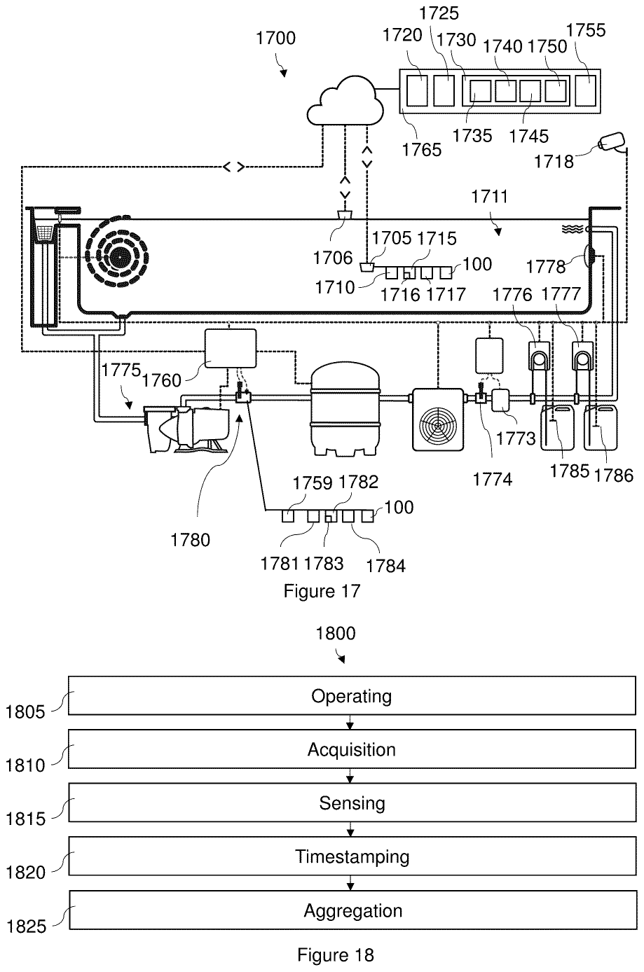

FIG. 17 represents, schematically, a particular embodiment of the system 1700 object of the present invention. This aquatic installation four-dimensional monitoring system 1700 comprises:
a submersible and/or floating vehicle, 1705 and 1706, comprising:
   a relative positioning coordinates acquisition means 1710, configured to locate, in a three-dimensional space representative of the aquatic installation 1711, the submersible vehicle and to provide the corresponding coordinates of the submersible and/or floating vehicle, and
   at least one physical and/or chemical sensor 1715, configured to provide series of at least one sensed value representative of a local physical/chemical parameter in the proximity of the submersible and/or floating vehicle,
a timestamping means 1720, configured to associate, with relative positioning coordinates acquired, a value representative of the time of acquisition, and
an aquatic installation physical and/or chemical state information aggregation means 1725, configured to associate timestamped coordinates to a local measured physical and or chemical state.

In particular embodiments, the system 1700 comprises at least one external sensor 1718, configured to provide an external graphical representation and/or video and/or picture acquisition of water in the aquatic installation and/or the aquatic installation and/or to provide external factors and/or parameters acquisition.

The submersible and/or floating vehicle, 1705 and/or 1706, may correspond, for example, to any manually, remotely and/or automatically dirigible vehicle adapted to the particular use case.

The vehicle 1705 may correspond to a remotely or autonomously dirigible submarine vehicle, for example, such as shown in FIG. 11.

The vehicle 1706 may also correspond to a floating pod, which is not dirigible, as shown in FIG. 12.

In particular embodiments, such as the one shown in FIG. 17, the system 1700 comprises both a submersible vehicle (ROV) 1705 and a floating vehicle 1706.

In particular variants, at least one submersible and/or floating vehicle, 1705 and/or 1706, comprises a solar panel 1105 configured to power an autonomous electricity source (not represented) and/or to charge the onboarded batteries (not represented).

In particular variants, at least one submersible and/or floating vehicle, 1705 and/or 1706, comprises an induction current collector configured to power an autonomous electricity source (not represented).

In particular variants, at least one submersible and/or floating vehicle, 1705 and/or 1706, comprises a power inlet configured to be connected to a charging wire to power an autonomous electricity source (not represented).

In particular variants, at least one submersible and/or floating vehicle, 1705 and/or 1706, comprises a propulsion 1110 system, such as an engine associated with a boat propeller. Such a propulsion 1110 system allows for the vehicle, 1705 and/or 1706, to move around in the water of the aquatic installation 1711. This propulsion system 1110 may comprise rear propellers 1120, configured to generate forward, backward or yaw movements, and a front propeller 1115, configured to generate upward or downward movements.

Such a submersible and/or floating vehicle, 1705 and/or 1706, further comprises a physical and/or chemical sensor 1715. Such a physical and/or chemical sensor 1715 is intended in the broadest sense, meaning that any physical and/or chemical parameter sensing device is encompassed, provided the output data of such a device is used to evaluate the physical and/or chemical state of the water in the basin 1711, of basin 1711 itself or of any other part of the aquatic installation.

The data provided by the physical/chemical sensor 1715 may also be complemented by data originating from at least one further physical and/or chemical sensor, 1781, 1782, 1783 and/or 1784, is situated in an analysis chamber 1780, or in a pipe of a circulation system where water flows, interacting with water in at least one aquatic installation and configured to provide series of at least one sensed value representative of a physical/chemical parameter. Such a physical/chemical parameter may be representative of a state of operation of the aquatic installation and/or of the water in the aquatic installation 1711.

The physical and/or chemical sensor, 1710, 1715, 1716, 1717, 1759, 1781, 1782, 1783 and/or 1784, is intended in the broadest sense, meaning that any physical and/or chemical parameter sensing device is encompassed, provided the output data of such a device is used to evaluate the physical and/or chemical state of the water in an aquatic installation 1711 and/or the physical and/or chemical state of the aquatic installation 1711, and/or status of equipment in the aquatic installation.

Such a water physical and/or chemical sensor, 1710, 1715, 1716, 1717, 1759, 1781, 1782, 1783 and/or 1784, may correspond to, but is not limited to:
a pH sensor, and/or
a total alkalinity sensor, and/or
a conductivity sensor, and/or
an oxidation-reduction potential sensor, and/or
a free chlorine sensor, and/or
a total chlorine sensor, and/or
a disinfectant rate sensor, and/or
a turbidity sensor, and/or
an optical sensor, and/or
a camera and/or video camera, and/or
an infrared sensor, and/or
an acoustic and/or sonar sensor, and/or
a temperature sensor, and/or
a flow sensor, and/or
a water movement sensor, and/or
a pressure sensor, and/or
a bacterial and/or algae activity sensor, and/or
a phosphate sensor, and/or
a nitrogen compounds sensor, and/or
a chloride sensor.

In particular embodiments, at least one water physical and/or chemical sensor 1715 is an image acquisition means configured to acquire an image in which a color is representative of a local chemical state of the aquatic installation.

Such sensors may be used to provide an environmental context of data acquisition by the water physical and/or chemical sensor 1715.

Such a submersible and/or floating vehicle, 1705 and/or 1706, further comprises a relative positioning coordinates acquisition means 1710, configured to locate, in a three-dimensional space representative of the aquatic installation 1711, the submersible vehicle, 1705 and/or 1706, and to provide the corresponding coordinates of the submersible and/or floating vehicle, 1705 and/or 1706.

Such a relative positioning coordinates acquisition means 1710 is, for example, a sonar configured to provide distance values from edges of the aquatic installation 1711. The distance values allow for the determination of the shape of the aquatic installation 1711. Once the shape of the aquatic installation 1711 is known, such distance values allow for the determination of the positioning of the submersible and/or floating vehicle, 1705 and/or 1706, within said installation 1711.

In another variant, the relative positioning coordinates acquisition means 1710 is, for example, a mechanical sensor used in coordination with a propulsion system 1110 to map the shape of the aquatic installation 1711 by detecting collisions of the submersible and/or floating vehicle, 1705 and/or 1706, with the edges of this installation 1711.

Once the shape of the aquatic installation 1711 is known, information originating from original parameters of the propulsion system 1110 may be used to locate the submersible and/or floating vehicle, 1705 and/or 1706. For example, a duration of use of the propulsion system 1110, associated with a power of propulsion, may be used in a calculation to determine a distance of the submersible and/or floating vehicle, 1705 and/or 1706, from the last known location.

The data resulting from the water physical and/or chemical sensor 1715 and the relative positioning coordinates acquisition means 1710 may be aggregated to form a timestamped water physical and/or chemical value sensed. This data may further be associated with environmental context values, such as water pressure or time of capture for example.

The timestamping means 1720 may correspond, for example, to any electronic clock used by a computing device. Such a timestamping means 1720 may be integrated into the submersible and/or floating vehicle, 1705 and/or 1706, or be remotely located from said submersible and/or floating vehicle, 1705 and/or 1706. By remotely located, it is intended that the timestamping means 1720 is linked with the submersible and/or floating vehicle, 1705 and/or 1706, by a communication means, such as a peer-to-peer link or a communications network link, such as the Internet for example.

The aquatic installation local physical and/or chemical state information aggregation means 1725 is, for example, a computer software executed upon a computing device such as illustrated in FIG. 14. This computing device is configured to associate, in a memory, the data resulting from the relative positioning coordinates acquisition means 1710, the water physical and/or chemical sensor 1715 and the timestamping means 1720.

Such an association may be performed by concatenating said data in a single data stream or data frame or by creating a link between said data if such data is stored in separate database tables for example.

In particular variants, the submersible and/or floating vehicle, 1705 and/or 1706, comprises the aquatic installation local physical and/or chemical state information aggregation means 1725.

In other variants, the aquatic installation local physical and/or chemical state information aggregation means 1725 is remotely located from the submersible and/or floating vehicle, 1705 and/or 1706, and accessible via a communication means.

In particular embodiments, such as the one represented in FIG. 17, the system 1700 object of the present invention comprises an aquatic installation physical parameter determination means 1730, configured to determine a value representative of a parameter of the aquatic installation or of the water in said installation as a function of several aggregated aquatic installation local physical and/or chemical state information.

This aquatic installation parameter determination means 1730 may be, for example, a computer software executed upon a computing device such as illustrated in FIG. 14. This aquatic installation parameter determination means 1730 may use an algorithmic module or a machine learning module to link a parameter value to at least one value sensed by the submersible and/or floating vehicle, 1705 and/or 1706, and/or by any other sensor associated with the aquatic installation 1711 and/or by any other sensor associated with a hydraulic circuit 1775 associated with the aquatic installation 1711.

An algorithmic module comprises a series of mathematical operations to be performed on a set or stream of data whereas a machine learning module comprises a machine learning architecture used on a training set or stream of data in order to produce a trained machine learning model that may then be used with operational data.

Such data represents, for example, physical and/or chemical sensed values, associated with time of measurement of said values.

In particular embodiments, such as the one shown in FIG. 17, the aquatic installation parameters determination means 1730 comprises an aquatic installation physical and/or chemical state uniformity determination means 1735, configured to determine a value representative of a physical and/or chemical state uniformity of the water in the aquatic installation 1711 as a function of several aggregated aquatic installation local chemical state information.

In such embodiments, the aim is to have the same values in several locations in the installation. In the aquatic installation water, there is always a different of values for any chemical parameters (such as pH, total alkalinity, disinfectant rate, etc.) linked to the water flow (area with a very low or almost no flow), the pollution content brought by users in the pool, and the products consumption. These differences generated concentration gradients which results in badly treated zones in the pool. Where total alkalinity is low (link to evaporation, or highly agitated water) the pH is less stable, and pH differences will occur. Where pH is higher than a set point (if we consider the set point to represent a pH at its water balance equilibrium value), the water step by step goes into a scaling state, which creates cloudy white water and scale deposition in the basin, pipes and equipment. In this case, chlorine will lose efficacy, and the area (mainly the surface) will be less disinfected.

On the contrary, when pH is lower than a set point (if we consider the set point to represent a pH at its water balance equilibrium value), the water step by step goes into an aggressive state, which damages the coating, and any equipment. In this case, the disinfectant has a higher efficacy, which could result in an extra oxidation level, creating attacks on coating and equipment.

In the case of high pollution brought by users, the disinfectant is consumed faster and generates disinfectant residues, which could become in excess if the disinfectant rate is low, or if its efficacy is not high enough. When in excess, these residues create irritations for users.

When the disinfectant rate is low, and its efficacy is low too, this helps the development of micro-organisms which could affect the health of users.

With the present system, it is possible to map the water in order to compensate any lack or excess of chemical rate to improve locally the water treatment. As an example, the lack of disinfectant could be compensated by adjusting the pump flow rate and/or injecting more disinfectant to help the good repartition of the disinfectant in the pool water and/or keep a perfect disinfection rate everywhere in the pool.

The term “physical and/or chemical state uniformity” refers to a measure of the variation in local physical and/or chemical state of the water in the aquatic installation 1711. Such a variation may correspond to a gradient, for example. The less variation is detected, the higher the determined uniformity value is. For example, a physical and/or chemical state uniformity value of one may correspond to a ratio of maximum to minimum pH values of less than two for two different areas of the water in the aquatic installation 1711.

The aquatic installation physical and/or chemical state uniformity determination means 1735 may be, for example, a computer software executed upon a computing device such as illustrated in FIG. 14.

In particular embodiments, such as the one shown in FIG. 17, the aquatic installation parameter determination means 1730 comprises a physical and/or chemical diffusion uniformity determination means 1740, configured to determine a value representative of physical parameter and/or of density uniformity of a chemical compound in the aquatic installation 1711 as a function of several aggregated aquatic installation local physical and/or chemical state information.

The term “diffusion uniformity” refers to a measure of the variation in local physical value and/or chemical composition of the water in the aquatic installation 1711. Such a variation may correspond to a gradient, for example. This term may also refer to the local variation of concentration of a specific or a group of chemical compounds within the water of an installation 1711. The less variation is detected, the higher the determined uniformity value is. For example, a uniformity value of one may correspond to a ratio of maximum to minimum concentration values of less than two for a specific chemical compound locally found in two different areas of the water in the aquatic installation 1711, for example, or to a ratio of operating parameters (temperature, water clarity) of the installation.

The physical and/or chemical diffusion uniformity determination means 1740 may be, for example, a computer software executed upon a computing device such as illustrated in FIG. 14.

In particular embodiments, such as the one shown in FIG. 17, the aquatic installation parameter determination means 1730 comprises a physical value and/or chemical compound flow determination means 1745, configured to determine a value representative of the flow of a physical and/or a chemical compound in the aquatic installation 1711 as a function of several aggregated aquatic installation local physical and/or chemical state information.

The term “flow” refers to a measure of a water physical parameter or chemicals in the aquatic installation 1711. Such a value for the flow may be obtained as a function of water pressure operated on the submersible and/or floating vehicle, 1705 and/or 1706. Such a value for the flow may be obtained as a function of the evolution, over time, in concentration of a specific physical parameter (such as temperature or turbidity) and/or chemical compound, group of chemical compounds, or chemical state (such as pH) in a set of locations in the aquatic installation 1711. Such a value may be performed based on a series of such measurements in several locations and for a series of chemicals added to the water of the aquatic installation 1711.

The physical parameter and/or chemical compound flow determination means 1745 may be, for example, a computer software executed upon a computing device such as illustrated in FIG. 14.

In particular embodiments, such as the one shown in FIG. 17, the aquatic installation physical parameter determination means 1730 comprises a risk zone determination means 1750, configured to determine a value representative of a risk relative to the local chemical state in at least part of the aquatic installation as a function of several aggregated aquatic installation local physical and/or chemical state information.

Such a risk zone may correspond to a difference for a measured parameter from a target value that exceeds a determined threshold value, such as, for example:
pH different from sent point (offset included), and/or
disinfectant rate different from sent point (offset included), and/or
total alkalinity different from sent point (offset included).

The risk zone determination means 1750 may be, for example, a computer software executed upon a computing device such as illustrated in FIG. 14.

In particular embodiments, a computing device, such as a remote computing device 1765, is configured to build and render a virtual representation of the aquatic installation 1711 and the aggregated data. This allows for providing a computer software which allows users to monitor the evaluation of the physical and/or chemical state of the water in the aquatic installation 1711 at different times. The use of specific color codes allows for providing advanced analytics, such as showing the presence of several physical parameters and/or chemical compounds in one representation of the water of the aquatic installation 1711, where each color is associated to a physical parameter value and/or to the presence and/or concentration of a distinct chemical compound.

In particular embodiments, such as the one shown in FIG. 17, the system 1700 object of the present invention comprises a command emitter 1755 configured to emit a command representative of a target operational value for an actuator 1760 interacting with the physical and/or chemical state of the aquatic installation.

The command emitter 1755 is, for example, a computer software executed upon a computing device, such as illustrated in FIG. 14, and associated to a communication means linking the emitter 1755 to the actuator 1760.

This command emitter 1755 is, for example, activated as a function of the result of a comparison between the value of a parameter sensed by the submersible and/or floating vehicle, 1705 and/or 1706, and a predetermined target value. Such a target value corresponds to, for example, a value representative of a desired physical and/or chemical state of the water in the aquatic installation 1711.

Such a command may correspond to, for example:
a release or the end of a release of a chemical compound in the water of the aquatic installation 1711, and/or
an increase or a decrease in a water pump activation, and/or
an increase or a decrease in water heater activation.

As it can be understood, in particular embodiments, such as the one shown in FIG. 17, the system 1700 object of the present invention may comprise a remote computing device 1765, comprising at least one of:
the timestamping means 1720, and/or
the information aggregation means 1725, and/or
the physical and/or chemical parameter determination means 1730.

The remote computing device 1765 is, for example, accessible in the cloud via communication means.

The remote computing device 1765 can be configured to control, register and adjust a set of predetermined installation 1711 operational parameters values (such as the pH or alkalinity for example).

The remote computing device 1765 may be, for example, operated as such:
all the data is routed by a traffic manager to a dedicated cloud (computing device and/or computer software):
   in each computing device and/or computing software, an algorithm manages the data's individual value from the camera acquisition of at least one vehicle, 1705 and/or 1706, and
   in a second step, an algorithm manages a combined global data value, and sends this data to a big data management module using the traffic manager, and
the global data from at least one vehicle, 1705 and/or 1706, and an external camera are compared to a user-set point:
   in case of differences between these values, the aquatic installation 1711 operational parameters are adjusted using water treatment and equipment piloting improvements, and
   in parallel, a timestamped three-dimensional mapping of the aquatic installation 1711 is recorded, and risk-determining algorithms are executed to determine the presence and severity of risks in the water installation 1711 a plurality of zones, and
   a timestamped three-dimensional map is generated comprising a color code for the pool risks to identify the risk prone zones in a simplified manner.

As it can be understood, in particular embodiments, such as the one shown in FIG. 17, the system 1700 object of the present invention may comprise at least a sensor 1759 and/or actuator 1760 configured to interact with an element 1780 of an installation sanitation circuit 1775 associated to the aquatic installation 1711, said sensor and/or actuator being activated as a function of at least one aggregated aquatic installation local physical and/or chemical state information.

Specifically, the element referenced 1780 refers to a chamber in which a sample of the water traversing the hydraulic circuit 1775 is analyzed by a sensor 1759. Such a sensor 1759 may correspond to an ORP sensor or alkalinity sensor. Such values may be compared with the values sensed by the submersible and/or floating vehicle, 1705 and/or 1706, and optionally adjusted with a determined offset value corresponding to a standard difference between a point of treatment and a point within the installation.

Another element may correspond to a filter 1771 analyzed by a sensor (not represented). Such a sensor may be adapted to provide values representative of the water pressure, or a level of dirt accumulated in the filter 1771. This filter 1771 may further be associated to an actuator (160). Such an actuator may be configured to automatically clean the filter. Such an actuator may be linked to a remote computing device 1765.

Another element may correspond to a heat pump 1772 associated to an actuator (160) configured to increase or decrease the temperature of the water. Such an actuator may be linked to a remote computing device 1765.

Another element may correspond to a disinfectant device 1773 (electro-chlorinator system, ultraviolet system, or ozone generator system or any possible in situ disinfectant generator) associated to an actuator (160) configured to increase or decrease the activation of the disinfectant device 1773. Such an actuator may be linked to a remote computing device 1765.

Such a disinfectant device 1773 may further be associated with an analysis chamber 1774 associated with a sensor (not referenced). Such a sensor may be configured to detect a level of bacterial activity in a sample of water from the aquatic installation 1711.

Another element may correspond to a pH adjustment device 1776 associated to an actuator (160) configured to increase or decrease the pH of the water. Such an actuator may be linked to a remote computing device 1765.

Such a pH adjustment device 1776 may be associated to a pH sensor (not represented) or a pH adjustment chemical compound drum level 1785.

Another element may correspond to a disinfectant (such as liquid chlorine or sodium hypochlorite or any other liquid disinfectant) release device 1777 associated to an actuator (160) configured to increase or decrease the concentration of disinfectant in the water. Such an actuator may be linked to a remote computing device 1765.

Such a disinfectant release device 1777 may be associated to a disinfectant chemical compound drum level 1786.

Another element may correspond to a light 1778 configured to illuminate the water in the basin 1711 and associated to an actuator (160) configured to activate or deactivate the light 1778. Such an actuator may be linked to a remote computing device 1765. The activation of such a light 1778 increases the performance of image-based sensors, such as particle sensors.

Another element may correspond to an installation cover 1779 configured to selectively cover the aquatic installation 1711.

This installation cover 1779 may be associated with an actuator configured to open or close the cover 1779, for purposes of safety, evaporation control, and energy cost reduction for example. Such an actuator may be linked to a remote computing device 1765.

This installation cover 1779 may be associated with a sensor (not referenced) configured to monitor the position of the cover 1779.

In particular embodiments, such as the one shown in FIG. 17, the system 1700 object of the present invention further comprises an external image capture device 1718, such as a video camera for example. This image capture device is configured to monitor the aquatic installation 1711 so as to provide additional data in relation to the water in the aquatic installation 1711 or the status of the system 1700, and notably the submersible (ROV) and/or floating vehicle, 1705 and/or 1706, within the aquatic installation 1711. Such data may correspond to infrared image capture, the detection of a number of bathers in the aquatic installation 1711, the presence of pollution in the aquatic installation 1711, a coating aspect of the aquatic installation 1711, a water clarity of the aquatic installation 1711 and position of the submersible and/or floating vehicle, 1705 and/or 1706.

In particular embodiments, the system 1700 comprises a total alkalinity measurement device, such as shown in FIG. 1.

FIG. 18 represents, schematically, a particular succession of steps of the method 1800 object of the present invention. This aquatic installation four-dimensional monitoring method 1800 comprises:
a step 1805 of operating a submersible and/or floating vehicle to navigate in an aquatic installation,
a step 1810 of relative positioning coordinates acquisition to locate, in a three-dimensional space representative of the aquatic installation, the submersible vehicle and to provide the corresponding coordinates of the submersible and/or floating vehicle,
a step 1815 of physical and/or chemical sensing to provide series of at least one sensed value representative of a local physical/chemical parameter in the proximity of the submersible and/or floating vehicle,
a step 1820 of timestamping to associate, with relative positioning coordinates acquired, a value representative of the time of acquisition and
a step 1825 of aquatic installation local physical and/or chemical state information aggregation to associate timestamped coordinates to a local measured physical and/or chemical state.

Particular embodiments of these steps are disclosed in regard to FIG. 17. As it can be understood:

FIG. 17 further represents a particular embodiment of the system object of the present invention, which comprises a total alkalinity regulation unit, configured to increase or decrease the total alkalinity of the body of water as a function of the measured total alkalinity value and a target total alkalinity value.

The total alkalinity regulation unit may correspond to any device suited for the regulation of alkalinity known to one skilled in the art. Such a device may be, for example, a mixer configured to mix acid from a reservoir and a stream of water processed in a swimming pool water treatment circuit.

The total alkalinity regulation unit operates to reach a target total alkalinity in the body of water. This target total alkalinity can be set by a user or be remotely set by a computing system. Such a value may be higher or equal to 120 mg/l (or ppm) and lower than 250 mg/l (or ppm). If the measured total alkalinity is above the target total alkalinity, the regulation unit may be operated to reduce the total alkalinity in the body of water whereas if the measured total alkalinity is below the target total alkalinity, the regulation unit may be operated to increase the total alkalinity in the body of water.

1. Aquatic total alkalinity measurement system, comprising:
a pH probe configured to measure pH at the boundary layer of a body of water, a probe controller, configured to sequentially activate and deactivate, or connect and disconnect, the pH probe,
a pH measurement variation detection device, configured to detect a variation of pH measurement in a sequence of pH probe measurements, and
an aquatic total alkalinity value determination device, configured to determine an aquatic total alkalinity value of the body of water as a function of the pH measurement variation detected.

2. System according to claim 1, which comprises a floating reference device in proximity of the pH probe.

3. System according to claim 2, in which the pH probe comprises:
the floating reference device,
a microporous glass bulb membrane, and
an oxidation-reduction potential sensor (135).

4. System according to claim 1, in which the pH probe controller is configured to sequentially activate and deactivate, or connect and disconnect, the pH probe in a stagnant body of water.

5. System according to claim 1, in which the pH probe is configured to be positioned in a low-volume body of water.

6. System according to claim 5, which comprises an analysis chamber, comprising a slotted opening, a main volume connected to the opening and a recess in the main volume, the pH probe being in contact with the water in the recess.

7. System according to claim 1, which comprises a remote computing device comprising the aquatic total alkalinity value determination device and a communication means between the pH measurement variation detection device and the aquatic total alkalinity value determination device.

8. System according to claim 1, in which the aquatic total alkalinity value determination device operates an algorithm and/or a trained machine learning model to associate an aquatic total alkalinity value with a variation in measured pH.

9. System according to claim 1, in which the pH probe is configured to measure the pH of the body of water in a swimming pool.

10. System according to claim 1, in which the pH probe is configured to measure the pH of the body of water in a pipe.

11. System according to claim 1, which comprises a total alkalinity regulation unit, configured to increase or decrease the total alkalinity of the body of water as a function of the measured total alkalinity value and a target total alkalinity value.

12. Aquatic total alkalinity measurement method, comprising:
a step of inserting, in a body of water, a pH probe configured to measure pH at the boundary layer of a body of water,
a step of sequential activation and deactivation, or connection and disconnection, of the pH probe,
a step of detection of pH measurement variation, to detect a variation of pH measurement in a sequence of pH probe measurements, and
a step of determination of an aquatic total alkalinity value, to determine an aquatic total alkalinity value of the body of water as a function of the pH measurement variation detected.
